(19) United States
(12) Patent Application Publication
(10) Pub. No.: US 2010/0183600 A1
(43) Pub. Date: Jul. 22, 2010

(54) RAF Inhibitors and Their Uses

(75) Inventors: Jean-Marc Lapierre, Pelham, NH (US); Yanbin Liu, Acton, MA (US); Manish Tandon, Framingham, MA (US); Mark A. Ashwell, Carlisle, MA (US)

Correspondence Address: Sunstein Kann Murphy & Timbers LLP, 125 SUMMER STREET, BOSTON, MA, 02110-1618, US

(73) Assignee: ARQULE, INC., Woburn, MA (US)

(21) Appl. No.: 12/631,579

(22) Filed: Dec. 4, 2009

Related U.S. Application Data
(60) Provisional application No. 61/120,198, filed on Dec. 5, 2008.

(51) Int. Cl. A61K 39/395; C07D 513/04; A61K 31/506; C07F 9/12; A61K 31/661; A61K 31/5685; A61K 33/24; A61K 31/7068; A61K 31/513; A61K 31/519; A61K 38/09; A61K 31/7048; A61K 31/704; A61K 38/12; A61K 31/5377; A61P 35/00

(52) U.S. Cl. 424/133.1; 544/331; 514/275; 544/243; 514/81; 514/171; 424/649; 514/110; 514/49; 514/274; 514/249; 514/15; 514/27; 514/34; 514/10; 514/234.5; 424/141.1

(57) ABSTRACT

The present invention provides imidazooxazole and imidazothiazole compounds and their syntheses. The compounds of the present invention are capable of inhibiting the activity of RAF kinase, such as B-RAF^{V600E}. The compounds are useful for the treatment of cell proliferative disorders such as cancer.

### PRIORITY

This patent application claims priority from provisional U.S. patent application No. 61/120,198, filed Dec. 5, 2008, entitled, “RAF INHIBITORS AND THEIR USES,” and naming Jean-Marc Lapierre, Yanbin Liu, Manish Tandon, and Mark A. Ashwell as inventors, the disclosure of which is incorporated herein, in its entirety, by reference.

### FIELD OF THE INVENTION

The invention generally relates to pharmaceutical compounds and compositions and, more particularly, the invention relates to inhibitors of RAF and uses thereof.

### BACKGROUND OF THE INVENTION

There are three RAF isoforms in humans: A-RAF, B-RAF and C-RAF (Marais and Marshall. Cancer Surv. 27:101-125 (1996)). These serine/threonine protein kinases are components of a conserved signaling pathway downstream of the membrane-bound small G protein RAS, which is activated by growth factors, hormones, and cytokines (Robinson and Cobb, Curr. Opin. Cell Biol. 9:180-186 (1997)). RAS stimulates RAF activation, which then leads to activation of the MEK kinase and subsequently the ERK kinase. Depending on the cellular context, this pathway mediates diverse biological functions such as cell growth, survival and differentiation predominantly through the regulation of transcription, metabolism and cytoskeletal rearrangements.

The RAS-RAF signaling pathway has long been associated with human cancers because oncogenic mutations in the ras gene occur in at least 15% of all human cancers (Davies, H. et al., Nature 417:949-954 (2002)), and the downstream kinase ERK is hyperactivated in 30% of cancers (Allen, et al., Semin. Oncol. 30:105-116 (2003)). However, for more than a decade, the RAF proteins had been considered to be important in cancer only because of their position downstream of RAS. This view was changed radically when activating mutations of B-RAF were found at a high frequency in human cancer, implicating B-RAF as a critical initiator and promoter of malignancy (Davies, H. et al., Nature 417:949-954 (2002)).

Activating mutations in the B-RAF protooncogene underlie 70% of melanomas, 50% of papillary thyroid cancers and 10% of colon cancers (Tuveson, et al., Cancer Cell 4:95-98 (2003); and Xing, Endocrine-Related Cancer: 12:245-262 (2005). Approximately 90% of these mutations occur as a single-nucleotide substitution that converts a valine to glutamate at amino acid 600 (V600E) in the kinase domain of B-RAF. This mutation increases the basal kinase activity of B-RAF, resulting in the activation of the MEK and ERK proteins that ultimately leads to uncontrolled tumor cell growth. Significantly, B-RAF and RAS mutations are usually mutually exclusive in the same tumor types, suggesting that these genes are on the same oncogenic signaling pathway and that RAS acts to activate B-RAF in these tumors.

Recent studies have found that knockdown of mutant B-RAF by small interference RNA in human melanoma cells inhibits both MEK and ERK kinases, causing growth arrest and ultimately promoting apoptosis (Sharma, et al., Cancer Res. 65:2412-2421 (2005); and Wellbrock et al., Cancer Res. 64:2338-2342 (2004)). In addition, data obtained from a short-hairpin RNA xenograft models targeting mutant B-RAF have shown that tumor regression resulting from B-RAF suppression is inducible, reversible, and tightly regulated (Hoeflich et al., Cancer Res. 66:999-1006 (2006). Taken together, gain-of-function B-RAF signaling is strongly associated with in vivo tumorigenicity, confirming B-RAF as an important target for cancer therapeutics.

The references cited herein are not admitted to be prior art to the claimed invention.

### SUMMARY OF THE INVENTION

An embodiment of the present invention provides a compound of formula I or pharmaceutically acceptable salts thereof

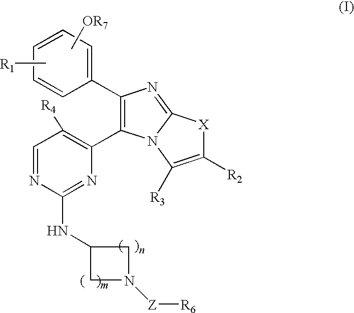

wherein

X is O, S(O)ₚ;

m is an integer from 1 to 3;

n is an integer from 1 to 3;

o is an integer from 0 to 2;

p is an integer from 0 to 2;

Z is hydrogen, a bond, —C(O)—, —C(O)NR₄—, —S(O)₂—;

R₁ is hydrogen, halogen, substituted or unsubstituted alkyl, —CN, —COOR₄, —OR₄, —NR₄R₅,

R₂ and R₃ are independently hydrogen, substituted or unsubstituted lower alkyl, —COOR₄, or —C(O)NR₄R₅;

each R₄ and each R₅ are independently hydrogen, substituted or unsubstituted alkyl, substituted or unsubstituted aryl, or substituted or unsubstituted heterocyclyl, and R₄ and R₅, taken together, may form a ring;

R₆ is independently selected from the group consisting of hydrogen, C₁-C₈ alkyl, C₁-C₈ fluoro-substituted alkyl, C₃-C₈ cycloalkyl, C₃-C₈ fluoro-substituted cycloalkyl, heterocyclyl, (C₁-C₈) alkyl-substituted heterocyclyl, aryl, halogen-substituted aryl, heteroaryl, and halogen-substituted heteroaryl;

R₇ is H or (CH₂O)ₒ—P(O)OR₄OR₅.

In an embodiment, R₂ and R₃ are hydrogen.

In an embodiment, R₄ is hydrogen.

In an embodiment, m+n=4, and if m is not equal to n, then the preferred stereochemical configuration is R.

In an embodiment, Z is hydrogen, a bond, —C(O)—, —C(O)NR₄—, —S(O)₂—; and R₆ is alkyl-substituted heterocyclyl, or alkyl-substituted heteroaryl.

In an embodiment, R₁ is hydrogen, halogen, substituted or unsubstituted alkyl, —CN, —COOR₄, —OR₄, —NR₄R₅,

In an embodiment, there is a compound selected from the group consisting of (R)-3-(5-(2-(1-(4-chlorophenylsulfonyl)piperidin-3-ylamino)pyrimidin-4-yl)imidazo [2,1-b]oxazol-6-yl)phenyl dihydrogen phosphate; (R)-3-(5-(2-(1-(4-chlorophenylsulfonyl)piperidin-3-ylamino)pyrimidin-4-yl)imidazo[2,1-b]thiazol-6-yl)phenyl dihydrogen phosphate; (R)-(3-(5-(2-(1-(4-chlorophenylsulfonyl)piperidin-3-ylamino)pyrimidin-4-yl)imidazo[2,1-b]thiazol-6-yl)phenoxy)methyl dihydrogen phosphate; (R)-(3-(5-(2-(1-(4-chlorophenylsulfonyl)piperidin-3-ylamino)pyrimidin-4-yl)imidazo[2,1-b]oxazol-6-yl)phenoxy)methyl dihydrogen phosphate; (R)-((3-(5-(2-(1-(4-chlorophenylsulfonyl)piperidin-3-ylamino)pyrimidin-4-yl)imidazo[2,1-b]thiazol-6-yl)phenoxy)methoxy)methyl dihydrogen phosphate; (3-(5-(2-(1-(4-chlorophenylsulfonyl)piperidin-4-ylamino)pyrimidin-4-yl)imidazo[2,1-b]thiazol-6-yl)phenoxy)methyl dihydrogen phosphate; (3-(5-(2-(1-(4-cyanophenylsulfonyl)piperidin-4-ylamino)pyrimidin-4-yl)imidazo[2,1-b]oxazol-6-yl)phenoxy)methyl dihydrogen phosphate; 3-(5-(2-(1-(4-fluorophenylsulfonyl)piperidin-4-ylamino)pyrimidin-4-yl)imidazo[2,1-b]oxazol-6-yl)phenyl dihydrogen phosphate; (3-(5-(2-(1-(cyclopropylsulfonyl)piperidin-4-ylamino)pyrimidin-4-yl)imidazo[2,1-b]oxazol-6-yl)phenoxy)methyl dihydrogen phosphate; ((3-(5-(2-(1-(cyclopropylsulfonyl)piperidin-4-ylamino)pyrimidin-4-yl)imidazo[2,1-b]oxazol-6-yl)phenoxy)methoxy)methyl dihydrogen phosphate; (3-(5-(2-(1-(1-methyl-1H-pyrazol-3-ylsulfonyl)piperidin-4-ylamino)pyrimidin-4-yl)imidazo[2,1-b]oxazol-6-yl)phenoxy)methyl dihydrogen phosphate; (R)-3-(5-(2-(1-(1-methyl-1H-pyrazol-3-ylsulfonyl)piperidin-3-ylamino)pyrimidin-4-yl)imidazo[2,1-b]oxazol-6-yl)phenyl dihydrogen phosphate; (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate; (R)-((3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methoxy)methyl dihydrogen phosphate; (R)-(3-(5-(2-(1-(1-methyl-1H-pyrazol-3-ylsulfonyl)piperidin-3-ylamino)pyrimidin-4-yl)imidazo[2,1-b]thiazol-6-yl)phenoxy)methyl dihydrogen phosphate; (R)-2-fluoro-5-(5-(2-(1-(1-methyl-1H-pyrazol-3-ylsulfonyl)piperidin-3-ylamino)pyrimidin-4-yl)imidazo[2,1-b]oxazol-6-yl)phenyl dihydrogen phosphate; and (R)-(2-fluoro-5-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate or a pharmaceutically acceptable salt thereof.

As embodiment of the present invention features the compound (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate or a pharmaceutically acceptable salt thereof.

An embodiment of the present invention features a prodrug, wherein the prodrug is hydrolyzed in vivo to give a compound of formula I as defined by claim 1, wherein R₇ is H or CH₂OH after the hydrolysis. In a related embodiment, R₇ is hydrogen or —(CH2O)—P(O)OR₄OR₅ before the hydrolysis.

An embodiment of the present invention also provides a pharmaceutical composition comprising a compound of formula I or a pharmaceutically acceptable salt thereof together with one or more pharmaceutically acceptable carriers or excipients. In an embodiment, the pharmaceutical composition further comprises a second chemotherapeutic agent. In related embodiments the second chemotherapeutic agent is selected from the group consisting of tamoxifen, raloxifene, anastrozole, exemestane, letrozole, cisplatin, carboplatin, paclitaxel, cyclophosphamide, lovastatin, mimosine, gemcitabine, Ara, 5-fluorouracil, methotrexate, docetaxel, goserelin, vincristine, vinblastine, nocodazole, teniposide, etoposide, epothilone, navelbine, camptothecin, daunorubicin, dactinomycin, mitoxantrone, amsacrine, doxorubicin, epirubicin, idarubicin imatanib, gefitinib, erlotinib, sorafenib, sunitinib malate, trastuzumab, rituximab, cetuximab, and bevacizumab. In another related embodiment the second chemotherapeutic agent is a taxane, an aromatase inhibitor, an anthracycline, a microtubule targeting drug, a topoisomerase poison drug, a targeted monoclonal or polyconal antibody, an inhibitor of a molecular target or enzyme (e.g., a kinase inhibitor), or a cytidine analogue drug In a further embodiment, the second chemotherapeutic agent is (−)-trans-3-(5,6-dihydro-4H-pyrrolo[3,2,1-ij] quinolin-1-yl)-4(1H-indol-3-yl)pyrrolidine-2,5-dione.

An embodiment of the present invention further provides a method of treating or preventing a cell proliferative disorder. The method comprises administering to a subject in need thereof a therapeutically effective amount of a compound of formula I, or a pharmaceutically acceptable salt thereof, in combination with a pharmaceutically acceptable carrier, wherein said cell proliferative disorder is treated.

In an embodiment, the cells with proliferative disorder contain DNA encoding a RAF, mutant or wild type. In a further embodiment, the cells have a constitutively enhanced RAF activity. The RAF can be A-RAF, B-RAF, or C-RAF. In an embodiment, B-RAF is a mutant; the mutant B-RAF can be B-RAF^{V600E}.

The cell proliferative disorder can be a precancerous condition, or a cancer. In an embodiment, the cell proliferative disorder is melanoma, papillary thyroid cancers, colon cancer, or Congenital Nevi.

The cell proliferative disorder may be a cancer including breast cancer, lung cancer, colorectal cancer, pancreatic cancer, ovarian cancer, prostate cancer, renal carcinoma, hepatoma, brain cancer, melanoma, multiple myeloma, chronic myelogenous leukemia, hematologic tumor, lymphoid tumor, sarcoma, carcinoma, and adenocarcinoma.

The present invention further provides a method of modulating B-RAF activity. The method comprises contacting a cell containing B-RAF gene with an effective amount of a compound of formula I, or a pharmaceutically acceptable salt thereof, analog or derivative thereof, wherein said contacting results in said inhibiting B-RAF activity. In an embodiment, The B-RAF activity is the kinase activity of B-RAF. In an embodiment, the B-RAF is B-RAF^{V600E}.

In an embodiment the method features administering the compound of formula I in combination with a second chemotherapeutic agent. In related embodiments, the second chemotherapeutic agent is one of tamoxifen, raloxifene, anastrozole, exemestane, letrozole, cisplatin, carboplatin, paclitaxel, cyclophosphamide, lovastatin, minocin, gemcitabine, Ara, 5-fluorouracil, methotrexate, docetaxel, goserelin, vincristin, vinblastin, nocodazole, teniposide, etoposide, epothilone, navelbine, camptothecin, daunorubicin, dactinomycin, mitoxantrone, amsacrine, doxorubicin, epirubicin, idarubicin imatanib, gefitinib, erlotinib, sorafenib, sunitinib malate, trastuzumab, rituximab, cetuximab, and bevacizumab.

In a related embodiment, the second chemotherapeutic agent is (−)-trans-3-(5,6-dihydro-4H-pyrrolo[3,2,1-ij] quinolin-1-yl)-4(1H-indol-3-yl)pyrrolidine-2,5-dione. For this combination, breast cancer, lung cancer, liver cancer, colon cancer or pancreatic cancer may be effectively treated.

An embodiment of the present invention features a method for manufacturing a medicament according to formula I for use in treating a cell proliferative disorder including the above-listed cancerous and precancerous conditions.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 shows a scheme for the synthesis of compounds of formula I.

FIG. 2 shows effects of compounds of formula I on Phospho-ERK in cancer cells.

FIG. 3 shows the effects of compounds of formula I on human tumors (A375) in a xenograft mouse model.

### DETAILED DESCRIPTION OF THE INVENTION

### 1. The Compounds

The present invention provides imidazooxazole and/or imidazothiazole compounds and their synthesis.

In an embodiment, the present invention provides compounds of formula I and their synthesis.

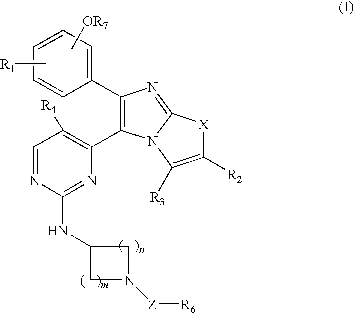

wherein

X is O, S(O)ₚ;

m is an integer from 1 to 3;

n is an integer from 1 to 3;

o is an integer from 0 to 2;

p is an integer from 0 to 2;

Z is hydrogen, a bond, —C(O)—, —C(O)NR₄—, —S(O)₂—;

R₁ is hydrogen, halogen, substituted or unsubstituted alkyl, —CN, —COOR₄, —OR₄, —NR₄R₅,

R₂ and R₃ are independently hydrogen, substituted or unsubstituted lower alkyl, —COOR₄, or —C(O)NR₄R₅;

each R₄ and each R₅ are independently hydrogen, substituted or unsubstituted alkyl, substituted or unsubstituted aryl, or substituted or unsubstituted heterocyclyl, and R₄ and R₅, taken together, may form a ring;

R₆ is independently selected from the group consisting of hydrogen, C₁-C₈ alkyl, C₁-C₈ fluoro-substituted alkyl, C₃-C₈ cycloalkyl, C₃-C₈ fluoro-substituted cycloalkyl, heterocyclyl, (C₁-C₈) alkyl-substituted heterocyclyl, aryl, halogen-substituted aryl, heteroaryl, (C₁-C₈) alkyl-substituted heteroaryl, and halogen-substituted heteroaryl;

R₇ is H or (CH₂O)ₒ—P(O)OR₄OR₅.

The term “alkyl” refers to radicals containing carbon and hydrogen, without unsaturation. Alkyl radicals can be straight or branched. Exemplary alkyl radicals include, without limitation, methyl, ethyl, propyl, isopropyl, hexyl, t-butyl, sec-butyl and the like. Alkyl groups may be denoted by a range, thus, for example, a (C₁-C₆) alkyl group is an alkyl group having from one to six carbon atoms in the straight or branched alkyl backbone. Substituted and unsubstituted alkyl groups may independently be (C₁-C₅) alkyl, (C₁-C₆) alkyl, (C₁-C₁₀) alkyl, (C₃-C₁₀) alkyl, or (C₅-C₁₀) alkyl. Unless expressly stated, the term “alkyl” does not include “cycloalkyl.” The term “lower alkyl” refers to unbranched or branched (C₁-C₆) alkyl.

A “cycloalkyl” group refers to a cyclic alkyl group having the indicated number of carbon atoms in the “ring portion,” where the “ring portion” may consist of one or more ring structures either as fused, spiro, or bridged ring structures. For example, a C₃ to C₆ cycloalkyl group (e.g., (C₃-C₆) cycloalkyl) is a ring structure having between 3 and 6 carbon atoms in the ring. When no range is given, then cycloalkyl has between three and nine carbon atoms ((C₃-C₉) cycloalkyl) in the ring portion. Exemplary cycloalkyl groups include, but are not limited to cyclopropyl, cyclobutyl, cyclopentyl, cyclohexyl, cycloheptyl, and adamantyl. Preferred cycloalkyl groups have three, four, five, six, seven, eight, nine, or from three to nine carbon atoms in the ring structure.

The term “aryl” refers to an aromatic carbocyclic group, having one, two, or three aromatic rings. Exemplary aryl groups include, without limitation, phenyl, naphthyl, and the like. Aryl groups include one, two, or three aromatic rings structures fused with one or more additional nonaromatic carbocyclic or heterocyclic rings having from 4-9 members. Examples of fused aryl groups include benzocyclobutanyl, indanyl, tetrahydronapthylenyl, 1,2,3,4-tetrahydrophenanthrenyl, tetrahydroanthracenyl, 1,4-dihydro-1,4-methanonaphthalenyl, benzodioxolyl.

The term “heteroaryl” refers to a heteroaromatic (heteroaryl) group having one, two, or three aromatic rings containing from 1-4 heteroatoms (such as nitrogen, sulfur, or oxygen) in the aromatic ring. Heteroaryl groups include one, two, or three aromatic rings structures containing from 1-4 heteroatoms fused with one or more additional nonaromatic rings having from 4-9 members. Heteroaryl groups containing a single type of hetroatom in the aromatic ring are denoted by the type of hetero atom they contain, thus, nitrogen-containing heteroaryl, oxygen-containing heteroaryl and sulfur-containing heteroaryl denote heteroaromatic groups containing one or more nitrogen, oxygen or sulfur atoms respectively. Exemplary heteroaryl groups include, without limitation, pyridyl, pyrimidinyl, pyrazolyl, triazolyl, quinolyl, quinazolinyl, thiazolyl, benzo[b]thiophenyl, furanyl, imidazolyl, indolyl, and the like.

The terms “heterocyclyl” or “heterocycle” refers to either saturated or unsaturated, stable non-aromatic ring structures that may be fused, spiro or bridged to form additional rings. Each heterocycle consists of one or more carbon atoms and from one to four heteroatoms selected from the group consisting of nitrogen, oxygen and sulfur. “Heterocyclyl” or “heterocycle” include stable non-aromatic 3-7 membered monocyclic heterocyclic ring structures and 8-11 membered bicyclic heterocyclic ring structures. A heterocyclyl radical may be attached at any endocyclic carbon or nitrogen atom that results in the creation of a stable structure. Preferred heterocycles include 3-7 membered monocyclic heterocycles (more preferably 5-7-membered monocyclic heterocycles) and 8-10 membered bicyclic heterocycles. Examples of such groups include piperidinyl, piperazinyl, pyranyl, pyrrolidinyl, morpholinyl, thiomorpholinyl, oxopiperidinyl, oxopyrrolidinyl, oxoazepinyl, azepinyl, isoxozolyl, tetrahydropyranyl, tetrahydrofuranyl, dioxolyl, dioxinyl, oxathiolyl, dithiolyl, sulfolanyl, dioxanyl, dioxolanyl, tetahydrofurodihydrofuranyl, tetrahydropyranodihydro-furanyl, dihydropyranyl, tetrahydrofurofuranyl, tetrahydropyranofuran, quinuclidinyl (1-azabicyclo[2.2.2]octanyl) and tropanyl (8-methyl-8-azabicyclo[3.2.1]octanyl).

The term substituted alkyl, substituted cycloalkyl, substituted aryl and substituted heterocyclyl refer to alkyl, cycloalkyl, aryl and heterocyclyl groups, as defined above, substituted with one or more substituents independently selected from the group consisting of fluorine, aryl, heteroaryl, —O—(C₁-C₆) alkyl, and —NR₅R₆, where R₅ and R₆ are independently selected from the group consisting of hydrogen and —(C₁-C₆) alkyl.

All stereoisomers of the compounds of the instant invention are contemplated, either in a mixture or in pure or substantially pure form, including crystalline forms of racemic mixtures and crystalline forms of individual isomers. The definition of the compounds according to the invention embraces all possible stereoisomers (e.g., the R and S configurations for each asymmetric center) and their mixtures. It very particularly embraces the racemic forms and the isolated optical isomers having a specified activity. The racemic forms can be resolved by physical methods, such as, for example, fractional crystallization, separation or crystallization of diastereomeric derivatives, separation by chiral column chromatography or supercritical fluid chromatography. The individual optical isomers can be obtained from the racemates by conventional methods, such as, for example, salt formation with an optically active acid followed by crystallization. Furthermore, all geometric isomers, such as E- and Z-configurations at a double bond, are within the scope of the invention unless otherwise stated. Certain compounds of this invention may exist in tautomeric forms. All such tautomeric forms of the compounds are considered to be within the scope of this invention unless otherwise stated. The present invention also includes one or more regioisomeric mixtures of an analog or derivative.

As used herein, the term “salt” is a pharmaceutically acceptable salt and can include acid addition salts including hydrochlorides, hydrobromides, in addition to salts formed by addition of a base such as phosphates, sulphates, hydrogen sulphates, alkylsulphonates, arylsulphonates, acetates, benzoates, citrates, maleates, fumarates, succinates, lactates, and tartrates. The salt may include alkali metal cations such as Na⁺, K⁺, Li⁺, alkali earth metal salts such as Mg²⁺ or Ca²⁺, or organic amine salts.

As used herein, the term “metabolite” means a product of metabolism of a compound of the present invention, or a pharmaceutically acceptable salt, analog or derivative thereof, that exhibits a similar activity in vivo to said compound of the present invention.

In an embodiment of the present invention, the compound is a compound of formula I wherein R₂ and R₃ are hydrogen.

In another embodiment of the present invention, the compound is a compound of formula I wherein R₄ is hydrogen.

In another embodiment of the present invention, the compound is a compound of formula I wherein R₁ is hydrogen, halogen, substituted or unsubstituted alkyl, —CN, —COOR₄, —OR₄, —NR₄R₅.

In related embodiments of the present invention, R₂ and R₃ are independently hydrogen, substituted or unsubstituted lower alkyl, —COOR₄, or —C(O)NR₄R₅.

In still other embodiments of the invention, each R₄ and each R₅ are independently hydrogen, substituted or unsubstituted alkyl, substituted or unsubstituted aryl, or substituted or unsubstituted heterocyclyl, and R₄ and R₅, taken together, may form a ring.

In related embodiments, R₆ is independently selected from the group consisting of hydrogen, C₁-C₈ alkyl, C₁-C₈ fluoro-substituted alkyl, C₃-C₈ cycloalkyl, C₃-C₈ fluoro-substituted cycloalkyl, heterocyclyl, (C₁-C₈) alkyl-substituted heterocyclyl, aryl, halogen-substituted aryl, heteroaryl, (C₁-C₈) alkyl-substituted heteroaryl, and halogen-substituted heteroaryl.

In another embodiment of the present invention, the compound is a compound of formula 1 wherein m+n=4, m is not equal to n, and the configuration is R. As used herein, the configuration of a molecule is the permanent geometry that results from the spatial arrangement of its atoms. The configuration can be either R or S and is defined according to the UIPAC rules. When more than one stereogenic atoms are present in a molecule, each one will be defined as of configuration R or S.

In another embodiment of the present invention, the compound is a compound of formula I wherein Z is hydrogen, a bond, —C(O)—, —C(O)NR₄—, —S(O)₂—; and R₆ is alkyl-substituted heterocyclyl, or alkyl-substituted heteroaryl.

In an embodiment of the present invention, the compound is one of compounds I-24 listed in table 1.

In an embodiment of the present invention, the compound is selected from the group consisting of (R)-3-(5-(2-(1-(4-chlorophenylsulfonyl)piperidin-3-ylamino)pyrimidin-4-yl)imidazo[2,1-b]thiazol-6-yl)phenyl dihydrogen phosphate; (R)-(3-(5-(2-(1-(4-chlorophenylsulfonyl)piperidin-3-ylamino)pyrimidin-4-yl)imidazo[2,1-b]thiazol-6-yl)phenoxy)methyl dihydrogen phosphate; (R)-3-(5-(2-(1-(4-chlorophenylsulfonyl)piperidin-3-ylamino)pyrimidin-4-yl)imidazo[2,1-b]oxazol-6-yl)phenyl dihydrogen phosphate; (R)-(3-(5-(2-(1-(4-chlorophenylsulfonyl)piperidin-3-ylamino)pyrimidin-4-yl)imidazo[2,1-b]oxazol-6-yl)phenoxy)methyl dihydrogen phosphate; (3-(5-(2-(1-(4-chlorophenylsulfonyl)piperidin-4-ylamino)pyrimidin-4-yl)imidazo[2,1-b]thiazol-6-yl)phenoxy)methyl dihydrogen phosphate; 3-(5-(2-(1-(4-fluorophenylsulfonyl)piperidin-4-ylamino)pyrimidin-4-yl)imidazo[2,1-b]oxazol-6-yl)phenyl dihydrogen phosphate; 3-(5-(2-(1-(cyclopropylsulfonyl)piperidin-4-ylamino)pyrimidin-4-yl)imidazo[2,1-b]oxazol-6-yl)phenyl dihydrogen phosphate; (3-(5-(2-(1-(cyclopropylsulfonyl)piperidin-4-ylamino)pyrimidin-4-yl)imidazo[2,1-b]oxazol-6-yl)phenoxy)methyl dihydrogen phosphate; (R)-3-(5-(2-(1-(1-methyl-1H-pyrazol-3-ylsulfonyl)piperidin-3-ylamino)pyrimidin-4-yl)imidazo[2,1-b]thiazol-6-yl)phenyl dihydrogen phosphate; (R)-(3-(5-(2-(1-(1-methyl-1H-pyrazol-3-ylsulfonyl)piperidin-3-ylamino)pyrimidin-4-yl)imidazo[2,1-b]thiazol-6-yl)phenoxy)methyl dihydrogen phosphate; (R)-3-(5-(2-(1-(1-methyl-1H-pyrazol-3-ylsulfonyl)piperidin-3-ylamino)pyrimidin-4-yl)imidazo[2,1-b]oxazol-6-yl)phenyl dihydrogen phosphate; (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate; (3-(5-(2-(1-(1-methyl-1H-pyrazol-3-ylsulfonyl)piperidin-4-ylamino)pyrimidin-4-yl)imidazo[2,1-b]oxazol-6-yl)phenoxy)methyl dihydrogen phosphate(R)-2-fluoro-5-(5-(2-(1-(1-methyl-1H-pyrazol-3-ylsulfonyl)piperidin-3-ylamino)pyrimidin-4-yl)imidazo[2,1-b]oxazol-6-yl)phenyl dihydrogen phosphate, or pharmaceutically acceptable salts thereof.

In another embodiment of the present invention, the compound is selected from the group consisting of (R)-3-(5-(2-(1-(4-chlorophenylsulfonyl)piperidin-3-ylamino)pyrimidin-4-yl)imidazo[2,1-b]thiazol-6-yl)phenyl dihydrogen phosphate; (R)-(3-(5-(2-(1-(4-chlorophenyl sulfonyl)piperidin-3-ylamino)pyrimidin-4-yl)imidazo[2,1-b]oxazol-6-yl)phenoxy)methyl dihydrogen phosphate; and (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate, or pharmaceutically acceptable salts thereof.

Certain embodiments include compounds of formula I that may serve as prodrug forms of the corresponding compounds of formulas I where R₇ is H. Without intending to be bound by the mechanistic explanation, the prodrug form may be cleaved by hydrolysis to release the corresponding compound where R₇ is H. The hydrolysis may occur by enzymatic or non-enzymatic routes that produce formula I where R₇ is H. Alternately, the hydrolysis may produce a corresponding hydroxymethylene derivative, which upon subsequent hydrolysis, may result in the release of compounds where R₇ is H. In one such embodiment R₇ is (CH₂O)ₒ—P(═O)OR₄OR₅, wherein o is 0-2. In a preferred embodiment, R₄ and R₅ are hydrogen. In a further preferred embodiment, o is 1, and R₄ and R₅ are hydrogen.

### 2. Methods and Intermediates for Preparing Compounds of the Invention

Standard synthetic methods and procedures for the preparation of organic molecules and functional group transformations and manipulations including the use of protective groups can be obtained from the relevant scientific literature or from standard reference textbooks in the field. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Wuts, P.G.M. Protective Groups in Organic Synthesis, 3R^{d}; John Wiley & Sons: New York, 1999. The following descriptions of synthetic methods are designed to illustrate, but not limit, general procedures for the preparation of compounds of the invention.

Compounds of the invention can be prepared in a variety of ways, some of which are known in the art. In general, the compounds of the present invention can be prepared from commercially available starting materials, compounds known in the literature, or from readily-prepared intermediates, by employing standard synthetic methods and procedures known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. The details for the synthesis of intermediates used in the present invention can be found in PCT patent publications WO 2004/110990, and WO 2006/044869, and WO 2007/123892.

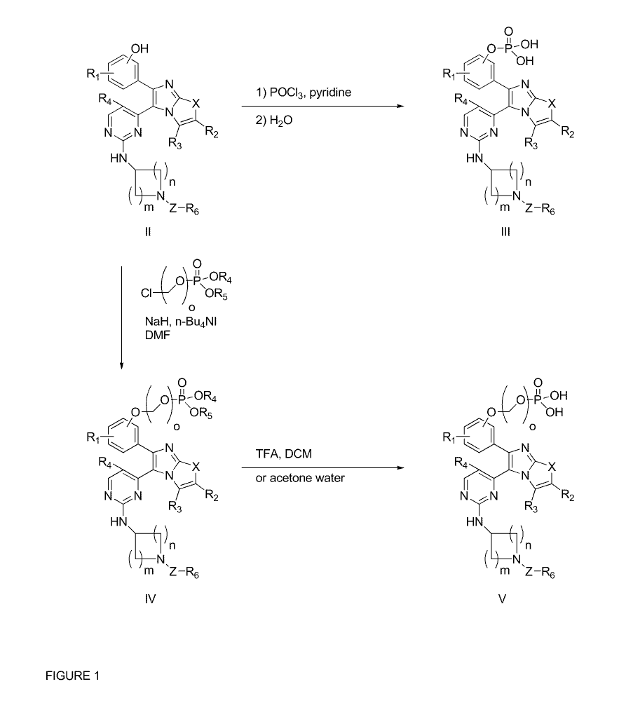

A method for preparing imidazooxazole and imidazothiazole compounds of the invention is described in the Examples below and illustrated in FIG. 1. In FIG. 1, intermediate II is reacted with phosphorus oxychloride in pyridine and quenched with water to provide the dihydrogen phosphate III. Alternatively, compound II is first deprotonated using sodium hydride in DMF, then treated with an appropriate chloromethyl phosphate in presence of tetrabutyl ammonium iodide to give intermediate IV. Conversion to the dihydrogen phosphate V is achieved using TFA in DCM or using milder conditions such as water in acetone at 40-50° C. The dihydrogen phosphate is optionally converted to the sodium or other pharmaceutically acceptable salt using aqueous sodium hydroxide or other bases.

### 3. Methods of Treatment

The compounds of the present invention can be used for the treatment and/or prevention of cell proliferative disorder such as cancer. The compounds of the present invention or a pharmaceutically acceptable salt or metabolite thereof, are capable of inhibiting one or more RAF protein kinases. Thus, the compounds can be used for the treatment of cell proliferative disorder characterized by aberrant RAS-RAF signaling. In an embodiment, the cells of cell proliferative disorder such as cancer harbor a mutated B-RAF. In a further embodiment, the mutated B-RAF is a B-RAF with the V600E mutation (B-RAF^{V600E}). The cell proliferative disorder can be melanomas, papillary thyroid cancers, colon cancers.

The present invention also provides a method of treating any other conditions characterized by a B-RAF^{V600E}, e.g., Congenital Nevi (commonly known as moles or freckles) possessing B-RAF^{V600E}, with the imidazooxazole and/or imidazothiazole compounds. In a further embodiment, the present invention may be used prophylactically (e.g., topically applied to the skin) to prevent such nevi to develop into malignant melanomas.

As used herein, a “subject” can be any mammal, e.g., a human, a primate, mouse, rat, dog, cat, cow, horse, pig, sheep, goat, camel. In a preferred aspect, the subject is a human.

As used herein, a “subject in need thereof” is a subject having a cell proliferative disorder, or a subject having an increased risk of developing a cell proliferative disorder relative to the population at large. In one aspect, a subject in need thereof has a precancerous condition. In a preferred aspect, a subject in need thereof has cancer.

As used herein, the term “cell proliferative disorder” refers to conditions in which unregulated or abnormal growth, or both, of cells can lead to the development of an unwanted condition or disease, which may or may not be cancerous. In one aspect, a cell proliferative disorder includes a non-cancerous condition, e.g., rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus. In another aspect, a cell proliferative disorder includes a precancer or a precancerous condition. In another aspect, a cell proliferative disorder includes cancer. Various cancers to be treated include but are not limited to breast cancer, lung cancer, colorectal cancer, pancreatic cancer, ovarian cancer, prostate cancer, renal carcinoma, hepatoma, brain cancer, melanoma, multiple myeloma, chronic myelogenous leukemia, hematologic tumor, and lymphoid tumor, including metastatic lesions in other tissues or organs distant from the primary tumor site. Cancers to be treated include but are not limited to sarcoma, carcinoma, and adenocarcinoma. In one aspect, a “precancer cell” or “precancerous cell” is a cell manifesting a cell proliferative disorder that is a precancer or a precancerous condition. In another aspect, a “cancer cell” or “cancerous cell” is a cell manifesting a cell proliferative disorder that is a cancer. Any reproducible means of measurement may be used to identify cancer cells or precancerous cells. In a preferred aspect, cancer cells or precancerous cells are identified by histological typing or grading of a tissue sample (e.g., a biopsy sample). In another aspect, cancer cells or precancerous cells are identified through the use of appropriate molecular markers.

A “cell proliferative disorder of the colon” is a cell proliferative disorder involving cells of the colon. In a preferred aspect, the cell proliferative disorder of the colon is colon cancer. In a preferred aspect, compositions of the present invention may be used to treat colon cancer or cell proliferative disorders of the colon. In one aspect, colon cancer includes all forms of cancer of the colon. In another aspect, colon cancer includes sporadic and hereditary colon cancers. In another aspect, colon cancer includes malignant colon neoplasms, carcinoma in situ, typical carcinoid tumors, and atypical carcinoid tumors. In another aspect, colon cancer includes adenocarcinoma, squamous cell carcinoma, and adenosquamous cell carcinoma. In another aspect, colon cancer is associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. In another aspect, colon cancer is caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

In one aspect, cell proliferative disorders of the colon include all forms of cell proliferative disorders affecting colon cells. In one aspect, cell proliferative disorders of the colon include colon cancer, precancerous conditions of the colon, adenomatous polyps of the colon and metachronous lesions of the colon. In one aspect, a cell proliferative disorder of the colon includes adenoma. In one aspect, cell proliferative disorders of the colon are characterized by hyperplasia, metaplasia, and dysplasia of the colon. In another aspect, prior colon diseases that may predispose individuals to development of cell proliferative disorders of the colon include prior colon cancer. In another aspect, current disease that may predispose individuals to development of cell proliferative disorders of the colon include Crohn's disease and ulcerative colitis. In one aspect, a cell proliferative disorder of the colon is associated with a mutation in a gene selected from the group consisting of p53, ras, FAP and DCC. In another aspect, an individual has an elevated risk of developing a cell proliferative disorder of the colon due to the presence of a mutation in a gene selected from the group consisting of p53, ras, FAP and DCC.

A “cell proliferative disorder of the skin” is a cell proliferative disorder involving cells of the skin. In one aspect, cell proliferative disorders of the skin include all forms of cell proliferative disorders affecting skin cells. In one aspect, cell proliferative disorders of the skin include a precancer or precancerous condition of the skin, benign growths or lesions of the skin, melanoma, malignant melanoma and other malignant growths or lesions of the skin, and metastatic lesions in tissue and organs in the body other than the skin. In another aspect, cell proliferative disorders of the skin include hyperplasia, metaplasia, and dysplasia of the skin.

In one aspect, a cancer that is to be treated has been staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) has been assigned a stage of MX, M0, or M1. In another aspect, a cancer that is to be treated has been staged according to an American Joint Committee on Cancer (AJCC) classification as Stage I, Stage IIA, Stage IIB, Stage IIIA, Stage IIIB, Stage IIIC, or Stage IV. In another aspect, a cancer that is to be treated has been assigned a grade according to an AJCC classification as Grade GX (e.g., grade cannot be assessed), Grade 1, Grade 2, Grade 3 or

Grade 4. In another aspect, a cancer that is to be treated has been staged according to an AJCC pathologic classification (pN) of pNX, pN0, PN0 (I−), PN0 (I+), PN0 (mol−), PN0 (mol+), PN1, PN1 (mi), PN1a, PN1b, PN1c, pN2, pN2a, pN2b, pN3, pN3a, pN3b, or pN3c.

In one aspect, a cancer that is to be treated includes a tumor that has been determined to be less than or equal to about 2 centimeters in diameter. In another aspect, a cancer that is to be treated includes a tumor that has been determined to be from about 2 to about 5 centimeters in diameter. In another aspect, a cancer that is to be treated includes a tumor that has been determined to be greater than or equal to about 3 centimeters in diameter. In another aspect, a cancer that is to be treated includes a tumor that has been determined to be greater than 5 centimeters in diameter. In another aspect, a cancer that is to be treated is classified by microscopic appearance as well differentiated, moderately differentiated, poorly differentiated, or undifferentiated. In another aspect, a cancer that is to be treated is classified by microscopic appearance with respect to mitosis count (e.g., amount of cell division) or nuclear pleiomorphism (e.g., change in cells). In another aspect, a cancer that is to be treated is classified by microscopic appearance as being associated with areas of necrosis (e.g., areas of dying or degenerating cells). In one aspect, a cancer that is to be treated is classified as having an abnormal karyotype, having an abnormal number of chromosomes, or having one or more chromosomes that are abnormal in appearance. In one aspect, a cancer that is to be treated is classified as being aneuploid, triploid, tetraploid, or as having an altered ploidy. In one aspect, a cancer that is to be treated is classified as having a chromosomal translocation, or a deletion or duplication of an entire chromosome, or a region of deletion, duplication or amplification of a portion of a chromosome.

In one aspect, a cancer that is to be treated is evaluated by DNA cytometry, flow cytometry, or image cytometry. In one aspect, a cancer that is to be treated has been typed as having 10%, 20%, 30%, 40%, 50%, 60%, 70%, 80%, or 90% of cells in the synthesis stage of cell division (e.g., in S phase of cell division). In one aspect, a cancer that is to be treated has been typed as having a low S-phase fraction or a high S-phase fraction.

As used herein, a “normal cell” is a cell that cannot be classified as part of a “cell proliferative disorder.” In one aspect, a normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “candidate compound” refers to a compound of the present invention that has been or will be tested in one or more in vitro or in vivo biological assays, in order to determine if that compound is likely to elicit a desired biological or medical response in a cell, tissue, system, animal or human that is being sought by a researcher or clinician. In one aspect, a candidate compound is a compound of formula I. In a preferred aspect, the biological or medical response is treatment of cancer. In another aspect, the biological or medical response is treatment or prevention of a cell proliferative disorder. In one aspect, in vitro or in vivo biological assays include, but are not limited to, enzymatic activity assays, electrophoretic mobility shift assays, reporter gene assays, in vitro cell viability assays.

As used herein, “monotherapy” refers to administration of a single active or therapeutic compound to a subject in need thereof. Preferably, monotherapy will involve administration of a therapeutically effective amount of an active compound. For example, cancer monotherapy with (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate comprises administration of a therapeutically effective amount of with (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate, or a pharmaceutically acceptable salt, analog or derivative thereof, to a subject in need of treatment of cancer. Monotherapy may be contrasted with combination therapy, in which a combination of multiple active compounds is administered, preferably with each component of the combination present in a therapeutically effective amount. In one aspect, monotherapy with a compound of the present invention is more effective than combination therapy in inducing a desired biological effect.

As used herein, “treating” describes the management and care of a patient for the purpose of combating a disease, condition, or disorder and includes the administration of a compound of the present invention to prevent the onset of the symptoms or complications, alleviating the symptoms or complications, or eliminating the disease, condition or disorder.

In one aspect, treating cancer results in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In another aspect, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In another preferred aspect, an increase in average survival time of a population may also be measured, for example, by calculating for a population the average length of survival following completion of a first round of treatment with an active compound.

In another aspect, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In another preferred aspect, an increase in average survival time of a population may also be measured, for example, by calculating for a population the average length of survival following completion of a first round of treatment with an active compound.

In another aspect, treating cancer results in increase in average survival time of a population of treated subjects in comparison to a population receiving monotherapy with a drug that is not a compound of the present invention, or a pharmaceutically acceptable salt, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In another preferred aspect, an increase in average survival time of a population may also be measured, for example, by calculating for a population the average length of survival following completion of a first round of treatment with an active compound.

In another aspect, treating cancer results in a decrease in the mortality rate of a population of treated subjects in comparison to a population receiving carrier alone. In another aspect, treating cancer results in a decrease in the mortality rate of a population of treated subjects in comparison to an untreated population. In a further aspect, treating cancer results a decrease in the mortality rate of a population of treated subjects in comparison to a population receiving monotherapy with a drug that is not a compound of the present invention, or a pharmaceutically acceptable salt, analog or derivative thereof. Preferably, the mortality rate is decreased by more than 2%; more preferably, by more than 5%; more preferably, by more than 10%; and most preferably, by more than 25%. In a preferred aspect, a decrease in the mortality rate of a population of treated subjects may be measured by any reproducible means. In another preferred aspect, a decrease in the mortality rate of a population may be measured, for example, by calculating for a population the average number of disease-related deaths per unit time following initiation of treatment with an active compound. In another preferred aspect, a decrease in the mortality rate of a population may also be measured, for example, by calculating for a population the average number of disease-related deaths per unit time following completion of a first round of treatment with an active compound.

In another aspect, treating cancer results in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured, for example, by measuring an increase in the diameter of a tumor after a prior tumor shrinkage that followed treatment. In another preferred aspect, a decrease in tumor regrowth is indicated by failure of tumors to reoccur after treatment has stopped.

In another aspect, treating or preventing a cell proliferative disorder results in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. In a preferred aspect, the rate of cellular proliferation is measured, for example, by measuring the number of dividing cells in a tissue sample per unit time.

In another aspect, treating or preventing a cell proliferative disorder results in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. In a preferred aspect, the proportion of proliferating cells is measured, for example, by quantifying the number of dividing cells relative to the number of nondividing cells in a tissue sample. In another preferred aspect, the proportion of proliferating cells is equivalent to the mitotic index.

In another aspect, treating or preventing a cell proliferative disorder results in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. In a preferred aspect, size of an area or zone of cellular proliferation may be measured as a diameter or width of an area or zone of cellular proliferation.

In another aspect, treating or preventing a cell proliferative disorder results in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. In one aspect, an abnormal cellular morphology is measured by microscopy, e.g., using an inverted tissue culture microscope. In one aspect, an abnormal cellular morphology takes the form of nuclear pleiomorphism.

As used herein, the term “selectively” means tending to occur at a higher frequency in one population than in another population. In one aspect, the compared populations are cell populations. In a preferred aspect, a compound of the present invention, or a pharmaceutically acceptable salt, analog or derivative thereof, acts selectively on a cancer or precancerous cell but not on a normal cell. In another preferred aspect, a compound of the present invention, or a pharmaceutically acceptable salt, analog or derivative thereof, acts selectively to modulate one molecular target (e.g., B-RAF). In another preferred aspect, the invention provides a method for selectively inhibiting the activity of an enzyme, such as a kinase. Preferably, an event occurs selectively in population A relative to population B if it occurs greater than two times more frequently in population A as compared to population B. More preferably, an event occurs selectively if it occurs greater than five times more frequently in population A. More preferably, an event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

In a preferred aspect, a compound of the present invention or a pharmaceutically acceptable salt, metabolite, analog or derivative thereof, modulates the activity of a molecular target (e.g., B-RAF). In one aspect, modulating refers to stimulating or inhibiting an activity of a molecular target. Preferably, a compound of the present invention modulates the activity of a molecular target if it stimulates or inhibits the activity of the molecular target by at least 2-fold relative to the activity of the molecular target under the same conditions but lacking only the presence of said compound. More preferably, a compound of the present invention modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

In one aspect, a compound of the present invention, or a pharmaceutically acceptable salt, metabolite, analog or derivative thereof, does not significantly modulate the activity of a molecular target if the addition of the compound does not stimulate or inhibit the activity of the molecular target by greater than 10% relative to the activity of the molecular target under the same conditions but lacking only the presence of said compound.

As used herein, the term “isozyme selective” means preferential inhibition or stimulation of a first isoform of an enzyme in comparison to a second isoform of an enzyme (e.g., preferential inhibition or stimulation of a kinase isozyme alpha in comparison to a kinase isozyme beta). Preferably, a compound of the present invention demonstrates a minimum of a four-fold differential, preferably a ten fold differential, more preferably a fifty fold differential, in the dosage required to achieve a biological effect. Preferably, a compound of the present invention demonstrates this differential across the range of inhibition, and the differential is exemplified at the IC₅₀, i.e., a 50% inhibition, for a molecular target of interest.

In a preferred embodiment, administering a compound of the present invention, or a pharmaceutically acceptable salt, metabolite, analog or derivative thereof, to a cell or a subject in need thereof results in modulation (i.e., stimulation or inhibition) of an activity of RAF. As used herein, an activity of RAF refers to any biological function or activity that is carried out by RAF. For example, a function of RAF includes phosphorylation of downstream target proteins.

In a preferred embodiment, administering a compound of the present invention, or a pharmaceutically acceptable salt, metabolite, analog or derivative thereof, to a cell or a subject in need thereof results in modulation (i.e., stimulation or inhibition) of an activity of ERK 1 or ERK 2, or both. As used herein, an activity of ERK 1 or ERK 2 refers to any biological function or activity that is carried out by ERK 1 or ERK 2. For example, a function of ERK 1 or ERK 2 includes phosphorylation of downstream target proteins.

In one aspect, activating refers to placing a composition of matter (e.g., protein or nucleic acid) in a state suitable for carrying out a desired biological function. In one aspect, a composition of matter capable of being activated also has an unactivated state. In one aspect, an activated composition of matter may have an inhibitory or stimulatory biological function, or both.

In one aspect, elevation refers to an increase in a desired biological activity of a composition of matter (e.g., a protein or a nucleic acid). In one aspect, elevation may occur through an increase in concentration of a composition of matter.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. In one aspect, a cell cycle checkpoint regulator is a protein. In another aspect, a cell cycle checkpoint regulator is not a protein.

In one aspect, treating cancer or a cell proliferative disorder results in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. In one aspect, number of cells in a population is measured by fluorescence activated cell sorting (FACS). In another aspect, number of cells in a population is measured by immunofluorescence microscopy. In another aspect, number of cells in a population is measured by light microscopy. In another aspect, methods of measuring cell death are as shown in Li et al., (2003) Proc Natl Acad Sci USA. 100(5): 2674-8. In an aspect, cell death occurs by apoptosis.

In a preferred aspect, an effective amount of a compound of the present invention, or a pharmaceutically acceptable salt, metabolite, analog or derivative thereof is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

In one aspect, contacting a cell with a compound of the present invention, or a pharmaceutically acceptable salt, metabolite, analog or derivative thereof, induces or activates cell death selectively in cancer cells. Preferably, administering to a subject in need thereof a compound of the present invention, or a pharmaceutically acceptable salt, metabolite, analog or derivative thereof, induces or activates cell death selectively in cancer cells. In another aspect, contacting a cell with a compound of the present invention, or a pharmaceutically acceptable salt, metabolite, analog or derivative thereof, induces cell death selectively in one or more cells affected by a cell proliferative disorder. Preferably, administering to a subject in need thereof a compound of the present invention, or a pharmaceutically acceptable salt, metabolite, analog or derivative thereof, induces cell death selectively in one or more cells affected by a cell proliferative disorder. In a preferred aspect, the present invention relates to a method of treating or preventing cancer by administering a compound of the present invention, or a pharmaceutically acceptable salt, metabolite, analog or derivative thereof to a subject in need thereof, where administration of the compound of the present invention, or a pharmaceutically acceptable salt, metabolite, analog or derivative thereof results in one or more of the following: accumulation of cells in G1 and/or S phase of the cell cycle, cytotoxicity via cell death in cancer cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3d ed.), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the invention.

In additional aspects, a compound of the present invention, or a pharmaceutically acceptable salt, metabolite, analog or derivative thereof, may be administered in combination with a second chemotherapeutic agent. The second chemotherapeutic agent can be a taxane, an aromatase inhibitor, an anthracycline, a microtubule targeting drug, a topoisomerase poison drug, a targeted monoclonal or polyconal antibody, an inhibitor of a molecular target or enzyme (e.g., a kinase inhibitor), or a cytidine analogue drug. In preferred aspects, the chemotherapeutic agent can be, but not restricted to, tamoxifen, raloxifene, anastrozole, exemestane, letrozole, HERCEPTIN® (trastuzumab), GLEEVEC® (imatinib), TAXOL® (paclitaxel), cyclophosphamide, lovastatin, mimosine, araC, 5-fluorouracil (5-FU), methotrexate (MTX), TAXOTERE® (docetaxel), ZOLADEX® (goserelin), vincristine, vinblastine, nocodazole, teniposide, etoposide, GEMZAR® (gemcitabine), epothilone, navelbine, camptothecin, daunorubicin, dactinomycin, mitoxantrone, amsacrine, doxorubicin (adriamycin), epirubicin or idarubicin or agents listed in the American Cancer Society's Guide to Cancer Drugs, available online; see, www.cancer.org/docroot/cdg/cdg_{—}0.asp. In another aspect, the second chemotherapeutic agent can be a cytokine such as G-CSF (granulocyte colony stimulating factor). In another aspect, a compound of the present invention, or a pharmaceutically acceptable salt, metabolite, analog or derivative thereof, may be administered in combination with radiation therapy. In yet another aspect, a compound of the present invention, or a pharmaceutically acceptable salt, metabolite, analog or derivative thereof, may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), or CMFP (cyclophosphamide, methotrexate, 5-fluorouracil and prednisone).

A compound of the present invention, or a pharmaceutically acceptable salt, metabolite, analog or derivative thereof, can be incorporated into pharmaceutical compositions suitable for administration. Such compositions typically comprise the compound (i.e. including the active compound), and a pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, ringer's solutions, dextrose solution, and 5% human serum albumin. Pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, ethylcellulose, hydroxypropylmethylcellulose, methylmethacrylate or the like. Other fillers, excipients, flavorants, and other additives such as are known in the art may also be included in a pharmaceutical composition according to this invention. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In one aspect, a compound of the present invention, or a pharmaceutically acceptable salt, metabolite, analog or derivative thereof, is administered in a suitable dosage form prepared by combining a therapeutically effective amount (e.g., an efficacious level sufficient to achieve the desired therapeutic effect through inhibition of tumor growth, killing of tumor cells, treatment or prevention of cell proliferative disorders, etc.) of a compound of the present invention, or a pharmaceutically acceptable salt, metabolite, analog or derivative thereof, (as an active ingredient) with standard pharmaceutical carriers or diluents according to conventional procedures (i.e., by producing a pharmaceutical composition of the invention). These procedures may involve mixing, granulating, and compressing or dissolving the ingredients as appropriate to attain the desired preparation.

### 4. The Pharmaceutical Compositions and Formulations

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount,” as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one aspect, the active compounds are prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 3000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

All patents, patent applications and references cited herein are incorporated by reference herein in their entirety.

### EXAMPLES

Examples are provided below to further illustrate different features of the present invention. The examples also illustrate useful methodology for practicing the invention. These examples do not limit the claimed invention.

### Example 1

### Preparation of bis-sodium (R) (3 (5 (2 (1-(1-methyl-1H-pyrazol-3-ylsulfonyl)piperidin-3-ylamino)pyrimidin-4-yl)imidazo[2,1-b]oxazol-6-yl)phenoxy)methyl phosphate

### Step 1: Preparation of Potassium di-tert-butyl Phosphate

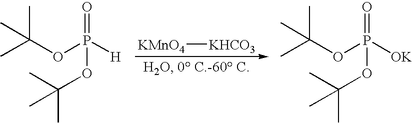

To a mixture of di-tert-butyl phosphonate (40.0 g, 206.2 mmol) and KHCO₃ (12.6 g) in water (178 ml) at 0° C. under vigorously stirring was added finely powdered KMnO₄ portionwise over 50 min (note; strongly exothermic reaction, efficient cooling is important). After addition, the mixture was stirred at room temperature for 30 min and then heated at 60° C. for 15 min. The by-product MnO₂ was filtered off Filtrate was decolorized by boiling with charcoal (3.2 g) and filtered. Filtrate was carried out to the next reaction without further purification.

### Step 2: Preparation of di-tert-butyl Hydrogen Phosphate

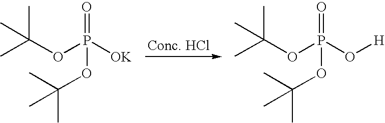

To the solution obtained from step 1 was added concentrated hydrochloric acid (16 ml) slowly at 0° C. with stirring. Product was precipitated out and collected by filtration, dried under vacuum overnight to provide 28.3 g of di-tert-butyl hydrogen phosphate as white needles.

### Step 3: Preparation of di-tert-butyl chloromethyl Phosphate

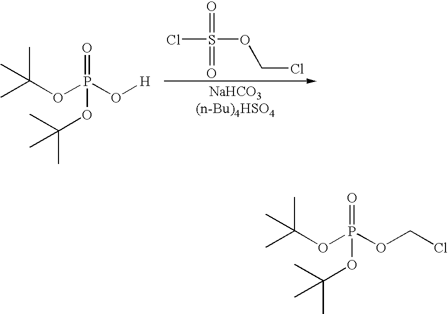

To a mixture of di-tert-butyl hydrogen phosphate (24.9 g, 133.3 mmol), NaHCO₃ (39.9 g, 533.3 mmol) and tetra-n-butylammonium hydrogen sulfate (4.0 g, 13.3 mmol) in water (1000 ml) was added dichloromethane (623 ml). The mixture was stirred at 0° C. for 20 min. and then a solution of chloromethyl chlorosulfate (23.5 g, 160 mmol) in dichloromethane (370 ml) was added with vigorously stirring. The resulting mixture was stirred at room temperature overnight (20 hs). Organic layer was separated, washed with brine (500 ml), dried over sodium sulfate and concentrated to provide 14.0 g of di-tert-butyl chloromethyl phosphate as a colorless oil. ¹H NMR (DMSO-d6) 400 MHz δ 5.72 (d, J=15.5 Hz, 2H), 1.14 (s, 18H).

### Step 4: Preparation of (R)-di-tert-butyl (3-(5-(2-(1-(1-methyl-1H-pyrazol-3-ylsulfonyl)-piperidin-3-ylamino)pyrimidin-4-yl)imidazo[2,1-b]oxazol-6-yl)phenoxy)methyl phosphate

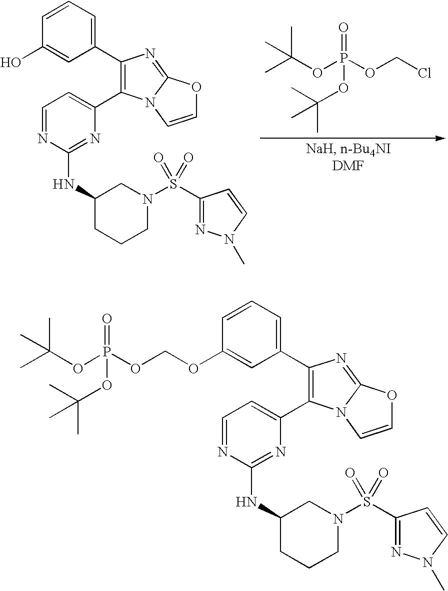

A mixture of (R)-3-(5-(2-(1-(1-methyl-1H-pyrazol-3-ylsulfonyl)piperidin-3-ylamino)pyrimidin-4-yl)imidazo[2,1-b]oxazol-6-yl)phenol (2.0 g, 3.85 mmol), sodium hydride (0.185 g, 7.69 mmol) and tetra-n-butyl ammonium iodide (0.42 g, 1.15 mmol) in N,N-dimethylformide (15 ml) was stirred at room temperature for 10 min. To this mixture was added a solution of di-tert-butyl chloromethyl phosphate (1.29 g, 5.0 mmol) in N,N-dimethylformide (5 ml). The resulting mixture was stirred at room temperature for 24 hs. Solvent was removed under vacuum. Residue was taken into dichloromethane (100 ml), washed with water (100 ml×2), dried over sodium sulfate and concentrated. Product was purified by flash column chromatography on silica gel to provide 1.60 g of (R)-di-tert-butyl (3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl phosphate as an orange solid. ¹H NMR (DMSO-d₆) 400 MHz δ 8.14-8.05 (m, 2H), 7.88 (d, J=2.6 Hz, 1H), 7.43 (t, J=8.1 Hz, 1H), 7.32-7.27 (m, 2H), 7.16-7.08 (m, 2H), 6.63 (d, J=2.2 Hz, 1H), 6.46 (d, J=5.1 Hz, 1H), 5.62 (d, J=11.7 Hz, 2H), 4.00-3.90 (m, 1H), 3.93 (s, 3H), 3.78-3.70 (m, 1H), 3.50-3.42 (m, 1H), 2.57 (br. t, J=10.1 Hz, 1H), 2.43 (br. t, J=10.3 Hz, 1H), 1.98-1.80 (m, 2H), 1.66-1.52 (m, 1H), 1.37 (s, 18H), 1.35-1.30 (m, 1H); LCMS M+H=743.

Alternative Method for Step 4

(R)-3-(5-(2-(1-(1-methyl-1H-pyrazol-3-ylsulfonyl)piperidin-3-ylamino)pyrimidin-4-yl)imidazo[2,1-b]oxazol-6-yl)phenol (31.56 g, 0.0606 mol, 1.0 equiv) and Cs₂CO₃ (39.49 g, 0.121 mol, 2.0 equiv) were charged into a flask. DMF (126 ml, 4 volumes) was added. The mixture was stirred at rt for 5 min. A solution of compound di-tert-butyl chloromethyl phosphate in DMF (63 ml, 3.7 volumes) was dropped into the mixture in 10 min. The resulting mixture was stirred at rt for 24 h. The reaction was complete (HPLC: 0.17% AUC of starting material). EtOAc (285 ml) was added. The mixture was cooled to 12° C. with vigorous stirring. Water (380 ml) was added in 10 min while keeping temperature below 22° C. The mixture was stirred for 10 min. The two layers were separated. The aqueous phase was extracted with EtOAc (285 ml). The combined organic phase was washed with brine (115 ml). The solution was evaporated to dryness to give a crude light red oil product (55 g, >100%,), without further purification for next step.

### Step 5: Preparation of (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate

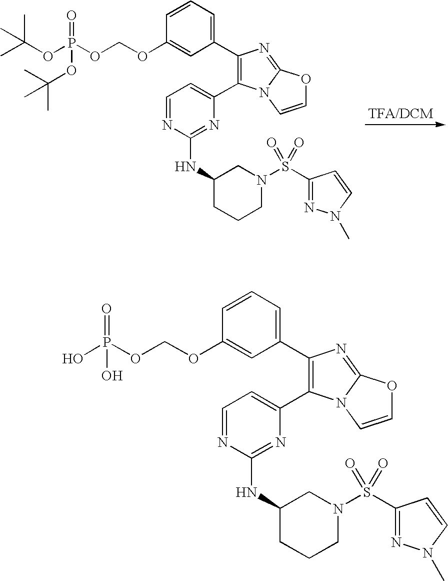

To a solution of (R)-di-tert-butyl (3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl phosphate (1.68 g, 2.26 mmol) in dichloromethane (34 ml) at 0° C. was added trifluoroacetic acid (2.61 ml, 34.0 mmol) dropwise. The mixture was stirred at room temperature for 45 min. (or until the disappearance of starting material). Solvent was removed under vacuum. Residue was stirred in ethyl ether (100 ml) for 2 hs. Product was collected by centrifuge and dried under vacuum overnight to provide 1.50 g of the title compound as an orange solid. The crude product was directly used in step 6 without purification. ¹H NMR (DMSO-d₆) 400 MHz δ 8.13-8.08 (m, 2H), 7.89 (d, J=2.2 Hz, 1H), 7.42 (t, J=7.9 Hz, 1H), 7.34-7.24 (m, 3H), 7.17-7.12 (m, 1H), 6.63 (d, J=2.2 Hz, 1H), 6.52 (d, J=5.5 Hz, 1H), 5.58 (d, J=11.7 Hz, 2H), 4.40-3.90 (m, 1H), 3.93 (s, 3H), 3.75-3.68 (m, 1H), 3.48-3.40 (m, 1H), 2.60 (br. t, J=10.1 Hz, 1H), 2.55-2.45 (m, 1H), 1.96-1.82 (m, 2H), 1.67-1.55 (m, 1H), 1.46-1.34 (m, 1H); LCMS M+H=631.

Alternative Method for Step 5

A solution of crude (R)-di-tert-butyl (3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl phosphate (28.15 g, 0.0326 mol,) in acetone (120 ml) was charged into 500 mL flask. Water (120 ml) was added with stirring. The cloudy mixture was heated to 50° C. for 18 h A white crystalline product precipitated. The mixture was then heated up to 55° C. for 24 h. The reaction was complete (monitored by HPLC). The reaction mixture cooled to 20° C., stirred for 3 h and filtered through a funnel. The cake was washed with water (3×120 ml) then washed with acetone (3×120 ml). The filter funnel was kept on the house vacuum for another 3 h. The greenish solid was dried in a vacuum oven (80° C./20 torr) for 6 h to afford the desired product (17.2 g).

### Step 6: Preparation of bis-sodium (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl phosphate

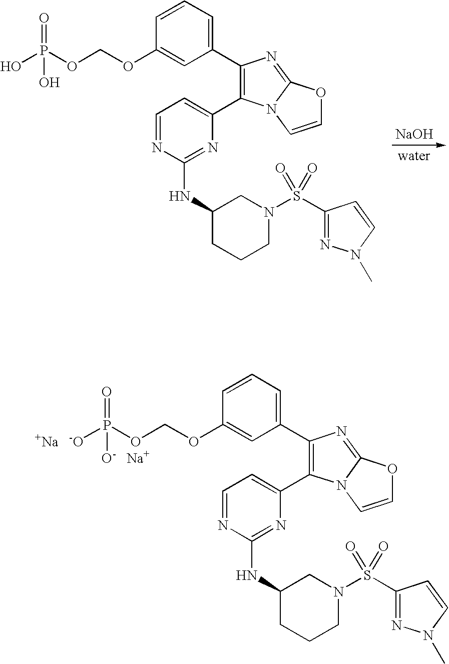

To (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate (2.93 g, 4.65 mmol) in a flask equipped with a stir bar was added a solution of sodium hydroxide (558 mg, 13.95 mmol) in water (30 ml). The resulting mixture was stirred until a clear solution was formed (30 min). The clear solution was transferred to a 500 ml flask. While stirring, 200 ml of acetone was added slowly. The resulting suspension was stirred for 10 min and then let it stand without stirring for 1 h. The liquid was decanted from the flask to waste. To the residue was added acetone (200 ml). The solution was vigorously stirred for 2 h. Solid product was collected by centrifuge. This solid was dissolved in 30 ml of water and 200 ml of acetone was added while stirring. After stirring for 10 min, the suspension was left without stirring for 2 h and the liquid was decanted. To the residue was added acetone (200 ml) and resulting mixture was stirred for 2 h. The solid was collected by centrifuge, dried under vacuum at 45° C. for 24 h to provide 2.30 g as an orange solid. ¹H NMR (D₂O) 400 MHz δ 7.63 (d, J=5.9 Hz, 1H), 7.59 (s, 1H), 7.54 (d, J=2.3 Hz, 1H), 7.40 (d, J=1.2 Hz, 1H), 7.22 (t, J=8.0 Hz, 1H), 7.10 (dd, J=8.2 2.0 Hz, 1H), 6.96 (s, 1H), 6.86 (d, J=7.8 Hz, 1H), 6.45 (s, 1H), 6.16 (d, J=5.5 Hz, 1H), 5.34 (d, J=8.6 Hz, 2H), 3.73 (s, 3H), 3.59-3.48 (m, 1H), 3.41 (br. d, J=9.4 Hz, 1H), 3.22 (br. d, J=11.3 Hz, 1H), 2.66-2.54 (m, 1H), 2.40-2.28 (m, 1H), 1.78-1.62 (m, 2H), 1.53-1.38 (m, 1H), 1.30-1.19 (m, 1H); LCMS M+H=631; Elemental analysis calculated for C₂₅H₂₄N₈O₈PS. 2.6 Na.0.6 TFA.0.4 acetone, 42.23% C, 3.54% H, 14.38% N, found, 42.22% C, 3.80% H, 14.27% N.

### Example 2

### Preparation of (R)-3 (5 (3 (1(4 chlorophenylsulfonyl)piperidin-3-ylamino)phenyl)imidazo[2,1-b]thiazol-6-yl)phenyl dihydrogen phosphate

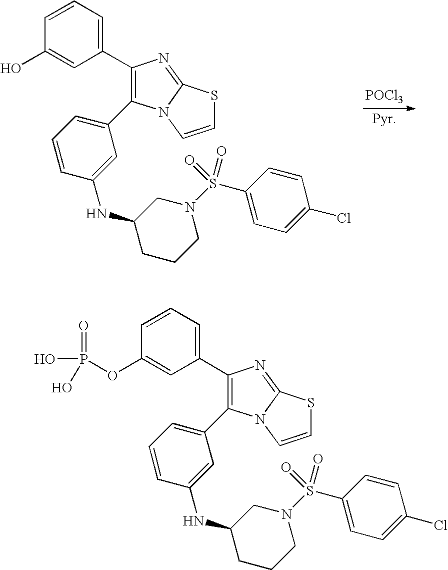

To a solution of (R)-3-(5-(3-(1-(4-chlorophenylsulfonyl)piperidin-3-ylamino)phenyl)imidazo-[2,1-b]thiazol-6-yl)phenol (0.322 g, 0.569 mmol) in pyridine (2.0 mL) at 0° C. was added POCl₃ (0.104 mL, 1.14 mmol). After addition, the mixture was stirred at room temperature for 2 hours, and then 2 mL of water was added. The resulting mixture was stirred overnight and acidified using 1 N HCl solution to PH=1-2. The solid was collected by centrifugation and purified by reverse phase HPLC using formic acid as a modifier. A yellow solid (110 mg) was obtained. M.p. 239-245° C.; ¹H NMR (DMSO-d₆) 400 MHz δ 8.70 (bs, 1H), 8.11 (d, J=5.6 Hz, 1H), 7.77-7.75 (m, 2H), 7.38-7.56 (m, 2H), 7.42 (m, 3H), 7.37-7.35 (m, 1H), 7.22-7.21 (m, 2H), 6.46 (d, J=5.2 Hz, 1H), 3.95 (bs, 1H), 3.72 (d, J=10 Hz, 1H), 3.46 (d, J=12 Hz, 1H), 2.52 (m, 1H), 2.34 (t, J=9.6 Hz, 1H), 1.87 (m, 2H), 1.60-1.57 (m, 1H), 1.43-1.37 (m, 1H); ³¹P NMR (DMSO-d₆) 400 MHz δ-5.201; LCMS M+H=647.

### Example 3

### Measurement of RAF Activity

Materials: The RAF kinases and the anti-phospho MEK1/2 antibody were from Upstate (Charlottesville, Va.). The RAF substrate used was full length N-terminal GST-MEK-1, which was expressed in E. coli and purified in-house by HPLC. All proteins were aliquoted and stored at −80° C. Superblock™ in phosphate buffered saline (PBS) blocking reagent was form Pierce (cat. #37515). ATP was from Roche (cat. # 19035722). Alkaline Phosphatase-tagged goat anti-rabbit antibody was from Pierce (cat. # 31340).

Methods: All RAF biochemical assays were performed using an assay buffer containing 20 mM MOPS, 5 mM EGTA, 37.5 mM MgCl2, 1 mM DTT and 50 μM ATP. There was 6.25 ng/well mutant B-RAF and 7.5 ng/well MEK-1 in the final assay conditions. Compounds were serially diluted in assay buffer containing 1% DMSO and 20 μl of test compound at a concentration 3-fold more than the final concentration, and were added to a polypropylene V-well reaction plate. Vehicle control wells received buffer only with DMSO at equivalent concentrations to the test wells. In rapid succession, 20 μl of substrate was added (0.45 ng/p1 MEK-1), followed by 20 μl of enzyme (0.375 ng/μl mutant B-RAF). These reaction plates were incubated at room temperature for 30 minutes. Capture of MEK-1 was initiated by transferring 50 μl of the reaction mixture to a Nunc Maxisorp™ microplate that is designed for non-specific protein capture. After 30 minute capture of MEK-1 at room temperature, this plate was washed with TBST (6×200 μL/well) to fully terminate the reaction. The plate was then blocked for 1 hour by the addition of 100 μl/well of Superblock™ in phosphate buffered saline (PBS) blocking reagent. Plate was again washed with TBST (6 times with 200 μl/well), followed by the addition of 70 μl/well of Upstate anti-phospho MEK ½ diluted 1:1000 in Pierce Superblock (PBS). After a 60 minute incubation, this plate was washed with TBST (6 times with 200 μl/well), and 70 μl of the secondary antibody (Pierce Alkaline Phosphatase tagged goat anti-rabbit) prepared at 1:4000 in Superblock, were added. After a 45 minute incubation at room temperature, the wells of the microplate were washed with TBST (6 times with 200 μl/well), and thereafter 100 μl/well of Attophos™ fluorescent alkaline phosphatase substrate was added according to the manufacturer's instructions (JBL Scientific). Fluorescence was read on a Perkin Elmer Envision multilabel reader, using the following filters: Excitation Filter: CFP430 nM, Emission Filter: Emission Filter 579 nM.

Compounds of the present invention prevent the phosphorylation of MEK through the inhibition of RAF kinases. The RAF/MEK/ERK pathway inhibition data for certain compounds of the present invention are shown in Table 1.

### Example 4

### Cell-Based Phosphorylation Assay with Electroblotting Readout

Compounds of the present invention have been screened for their ability to inhibit all isoforms, both wild-type and mutant of RAF kinases (A-RAF, B-RAF and C-RAF) in general, and the mutant B-RAF (V600E) in particular in human cancer cells. A375 is a human melanoma cell line that harbors the most common B-RAF mutation-V600E found in human cancers. The ability of compounds to inhibit RAF kinases in this assay is correlated with the reduction of MEK and ERK phosphorylation, and is therefore a direct indicator of potential in vivo therapeutic activity.

Materials: A375 cells from ATCC were maintained at 37° C., 5% CO2 in DMEM media supplemented with 10% fetal bovine serum, penicillin/streptomycin and fungizone. (Invitrogen)

Methods: Test compounds were dissolved and diluted 1:1000 in DMSO. A375 cells were seeded in six-well tissue culture plates at 5-8×10⁵ per well and cultured at 37° C. for 24 h. Cells were incubated with compounds for one hour before being lysed in EPage™ loading buffer (Invitrogen). Lysates were electrophoresed on 8% EPage™ gels and transferred to polyvinylidene difluoride membranes. After incubations with primary and secondary antibodies, the immunostained proteins were detected and quantitated by an Odyssey infrared imager (LI-COR). Analysis was performed by non-linear regression to generate a dose response curve. The calculated IC₅₀ value was the concentration of the test compound that causes a 50% decrease in phospho-MEK and phospho-ERK levels. The primary antibodies used were anti-MEK (Stressgen), anti-ERK (BD Biosciences), anti-phospho-ERK and anti-phospho-MEK (Cell Signaling). The secondary antibodies used were IRDYE800 anti-rabbit, IRDYE 800 anti-mouse (Rockland), AlexaFluor680 anti-mouse and AlexaFluoro680 anti-rabbit (Invitrogen).

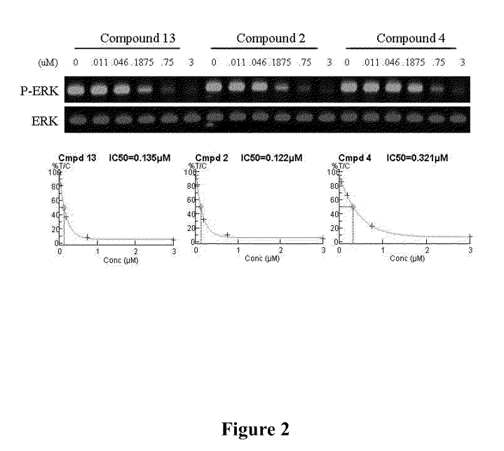

FIG. 2 shows effects of compounds of formula I on Phospho-ERK in cancer cells. A375 cells were treated with 0, 12, 37, 111, 333 and 1000 nM of indicated compounds for 1 hr. The levels of Phospho-ERK and total-ERK were accessed by immunoblotting.

Compounds of the present invention reduce the levels of phospho-MEK and phospho-ERK through the inhibition of RAF kinases. The RAF/MEK/ERK pathway inhibition data for certain compounds of the present invention are shown FIG. 2 and in Table 1.

### Example 5

### Cell Growth Inhibition Assay

Compounds of the present invention have been tested for activity against a variety of cancer cell lines. The ability of compounds to inhibit cell growth in this assay is correlated with the reduction of dehydrogenase enzyme activity found in metabolically active cells.

Materials: A large variety of cancer cells from ATCC were maintained at 37° C., 5% CO₂ in DMEM media supplemented with 10% fetal bovine serum, penicillin/streptomycin and fungizone (Invitrogen). NCM460 (Incell), a normal colon epithelial cell line, and human mammary epithelial cells (Cambrex) were maintained at 37° C., 5% CO₂ in DMEM and HEBM media (Cambrex), respectively.

Methods: Test compounds were dissolved and diluted to 300× in DMSO then diluted 1:40 in DMEM. Cells were seeded into 96-well tissue culture plates at 1-5×10³ per well and cultured at 37° C. for 24 h. Cells were incubated with test compounds for 72 hours followed by incubation with tetrazolium compound (3-(4,5-dimethylthiazol-2-yl)-5-(3-carboxymethoxyphenyl)-2-(4-sulfophenyl)-2H-tetrazolium, inner salt; MTS) and the electron coupling reagent, phenazine methosulfate (PMS) for 4 hr. MTS was chemically reduced by dehydrogenase in cells into formazan. The measurement of the absorbance of the formazan was assessed using an ENVISION™ (Perkin Elmer) microplate reader at 492 nm. The calculated IC₅₀ value is the concentration of the test compound that causes a 50% decrease in the absorbance.

Compounds of the present invention inhibit the growth of a variety of cancer cells. The data for certain compounds of the invention are shown in Table 2 and Table 3.

### Example 6

### Observations Regarding Patterns of Compound Activity

Due to unexpected patterns of enzymatic and cell-line inhibition, certain compounds of formula I are believed to be prodrugs. Furthermore, these compounds surprisingly exhibit dramatic increases in solubility thereby aiding the ability to formulate them into effective therapeutic compositions.

Compound 15 and 16 are analogues of compound 14 in that they share the common parent structure of compound 14 but have a phosphate or methyl phosphate linked at the phenolic oxygen of the parent structure. As shown in Table I, thiazole compounds 15 and 16 are far less potent in inhibiting mutant B-RAF than compound 14. In particular, compound 15 has about a 400 fold increase in IC₅₀ relative to compound 14 and compound 16 has about a 193-fold increase. However, a similar pattern does not appear for the cell-based ERK inhibition data, also shown in Table I, which deviate by about 2 and 4 fold in EC₅₀ respectively. A similar effect is seen with the oxazole analogues: compounds 17, 18 and 19. For example, compound 19 has a 365-increase in B-RAF IC₅₀ relative to compound 17 and almost no change in EC₅₀.

An explanation for the above discrepancy for in-vivo versus in vitro activity is that compounds 15, 16, 18 and 19 are acting as prodrugs that are metabolized or otherwise converted by the A375 cells to yield compounds 14 and 17, respectively.

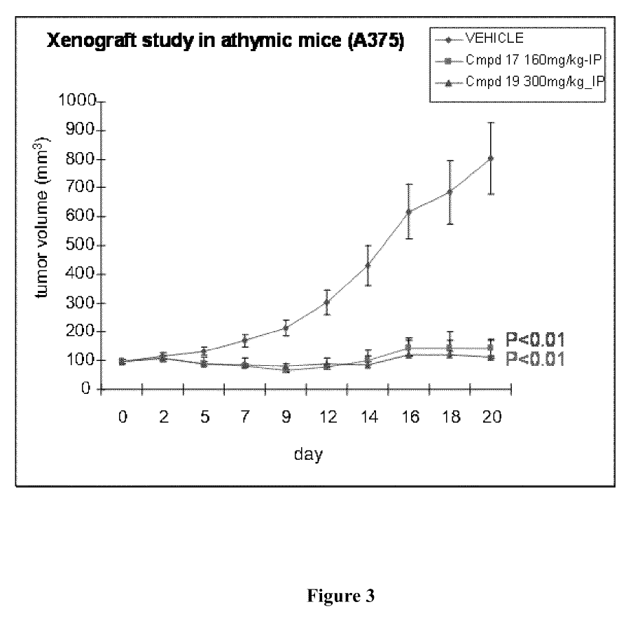

As shown in FIG. 3, compounds 17 and 19 retain their anti-tumor activity in a xenograft cancer model (measurement details given in Example 7). Compound 17 was injected intra-peritoneally (IP) at 160 mg/kg and compound 19 was injected IP at 300 mg/kg (equivalent dose if expressed in mmole/kg, taking into account salt components).

Although compounds 17 and 19 appear to be equally effective in the xenograft model of FIG. 3, compound 19 has further potential advantages. As can be seen in Table 4, compound 19 is dramatically more soluble in aqueous solution at or near biologically relevant pH. Thus, compound 19 is likely to be easier to formulate (e.g., for preparing an intravenous solution, or for other delivery methods known in the art). Details of the solubility measurements are given in Example 8.

### Example 7

### Protocol for Xenograft Model in Athymic Mice

A mouse xenograft model was performed according to the method of Jacob et al, Gene Ther Mol Biol 2004; 8:213-219 and Wilhelm et al, Cancer Research 2004; 64: 7099-7109.

Animal Care. Six week old female NCr nu/nu mice were purchased from Taconic Farms, Germantown, N.Y. and allowed to acclimate 1-2 weeks. Mice were housed in sterile micro isolator cages, 5 mice per cage and receive food and water ad libitum. All experimental procedures and surgical manipulations were approved in accordance with ArQule's Institutional Animal Care and Use Committee (IACUC).

Tumor Cell Lines and Model. Carcinoma cell lines were obtained from and propagated as recommended by American Type Tissue Culture (ATCC), (Manassas, Va.). Mice were implanted subcutaneously with 2.5-10×10⁶ cells in 0.1 ml sterile Hanks Balanced Salt Solution (HBSS) in the upper right flank area. Administration of compound began when tumor size ranged between 75 and 200 mg. Tumor measurements and body weights were collected two to three times a week with an electronic calipers and balance. Tumor weight (mg) was calculated from the equation length×(width)²)/2; this formula can also be used to calculate tumor volume assuming unit density 1 mg=1 mm³ Percent inhibition or tumor growth inhibition (TGI) was calculated using the following formula: 1-[mean tumor value of treated/mean tumor value of control]×100. Treatments producing >30% lethality and/or >20% net body weight loss can be considered toxic.

### Example 8

### Protocol for Equilibrium Solubility Determination at Varying pH

Solubility of the compounds was determined by the traditional shake flask method. Aliquots of solid compounds were mixed with an appropriate buffer of the desired pH and equilibrated by shaking at room temperature (˜25° C.) for 6-24 hours. Following aqueous buffers were used for the pH control: 0.1N HCl pH=1.2, 50 mM Lactate buffer pH=3.0, 50 mM Acetate buffer pH=5.0, and 100 mM Phosphate buffer pH 7.4. After equilibration, samples were filtered through a 0.45 um filter and analyzed by HPLC/UV against the calibration curves of standard solution.

### Example 9

### Combination of Exemplary Compounds of the Present Invention with c-Met Inhibitors

Unless otherwise stated, the following materials and methods apply to the biological assays described herein. Cell culture and reagents: Cancer cell lines were cultured in DMEM or RPMI medium containing 10% fetal bovine serum, 100 units/ml penicillin, 100 μg/ml streptomycin, and 2 mM L-glutamine.

Cell proliferation analysis. Cell survival was determined by the MTS assay. Briefly, cells were plated in a 96-well plate at 2,000-10,000 cells per well, cultured for 24 hours in complete growth medium, and then treated with various drugs and drug combinations for 72 hours. MTS was added and incubated for 4 hour, followed by assessment of cell viability using the microplate reader at 570 nm. Data were normalized to untreated controls and analyzed with Microsoft Excel.

The studies described herein used a compound of Formula I shown herein, namely, (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate, a V600E mutant-B-Raf inhibitor in combination with a small molecule inhibitor of the c-Met receptor tyrosine kinase, (−)-trans-3-(5,6-dihydro-4H-pyrrolo[3,2,1-ij] quinolin-1-yl)-4(1H-indol-3-yl)pyrrolidine-2,5,-dione.

A panel of 55 human cancer cell lines encompassing a spectrum of genotypes and tissue origins were surveyed in 72 h MTS cytotoxicity assays across a wide range of compound concentrations. The compounds (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate and (−)-trans-3-(5,6-dihydro-4H-pyrrolo[3,2,1-ij] quinolin-1-yl)-4(1H-indol-3-yl)pyrrolidine-2,5-dione were configured in checkerboard 3-fold dilutions for 72-hr MTS assay.

In the instant example, two independent experiments were performed in parallel. Chou algorithm employed to calculate Combination Index (CI) as shown below.

Criteria for Combination Index (CI)

| | |
|---|---|
| CI ≦ 0.3 | Strong Synergy |
| 0.3 < CI ≦ 0.85 | Synergy |
| 0.85 < CI ≦ 1.2 | Additive |
| 1.2 < CI ≦ 3.3 | Antagonism |
| CI ≧ 3.3 | Strong Antagonism |

The combination data of (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate with (−)-trans-3-(5,6-dihydro-4H-pyrrolo[3,2,1-ij] quinolin-1-yl)-4(1H-indol-3-yl) pyrrolidine-2,5-dione is shown in Table 5. The identity and tissue origin of cancer cell lines are indicated. The results show that the combination (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate with (−)-trans-3-(5,6-dihydro-4H-pyrrolo[3,2,1-ij] quinolin-1-yl)-4(1H-indol-3-yl) pyrrolidine-2,5-dione resulted in synergistic cytotoxicity in many cell lines including, NCI-H52 (NSCLC), MDA-MB-231 (breast), SNU475 (liver) and in PC3 (prostate) cell lines and showed additive cytotoxicity in many other cell lines.

TABLE 1
Exemplary Compounds of the Invention

| | | | | Mut-B- | p-Erk |
|---|---|---|---|---|---|
| | | | | Raf | EC₅₀ |
| | | | | IC₅₀ | (A375) |
| | Structure | Name | [M + H] | (μM) | (μM) |
| 1 | 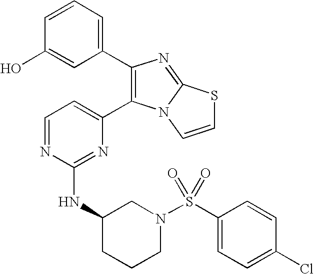 | (R)-3-(5-(2-(1-(4- chlorophenylsulfonyl)piper- idin-3-ylamino)pyrimidin- 4-yl)imidazo[2,1-b]thiazol- 6-yl)phenol | 567 | 0.006 | 0.011 |
| 2 | 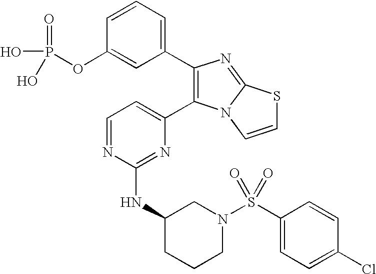 | (R)-3-(5-(2-(1-(4- chlorophenylsulfonyl)piper- idin-3-ylamino)pyrimidin- 4-yl)imidazo[2,1-b]thiazol- 6-yl)phenyl dihydrogen phosphate | 647 | 1.9 | 0.26 |
| 3 | 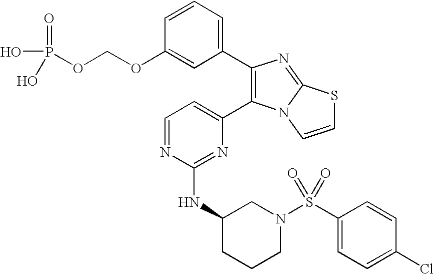 | (R)-(3-(5-(2-(1-(4- chlorophenylsulfonyl)piper- idin-3-ylamino)pyrimidin- 4-yl)imidazo[2,1-b]thiazol- 6-yl)phenoxy)methyl dihydrogen phosphate | 677 | 0.375 | 0.071 |
| 4 | 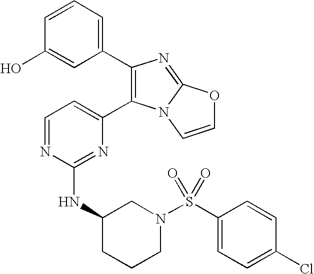 | (R)-3-(5-(2-(1-(4- chlorophenylsulfonyl)piper- idin-3-ylamino)pyrimidin- 4-yl)imidazo[2,1-b]oxazol- 6-yl)phenol | 551 | 0.01 | 0.035 |
| 5 | 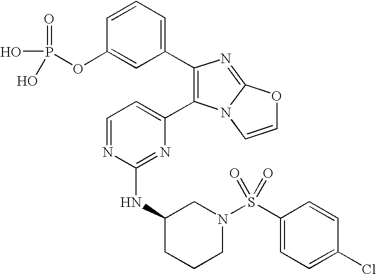 | (R)-3-(5-(2-(1-(4- chlorophenylsulfonyl)piper- idin-3-ylamino)pyrimidin- 4-yl)imidazo[2,1-b]oxazol- 6-yl)phenyl dihydrogen phosphate | 631 | 1.9 | 0.127 |
| 6 | 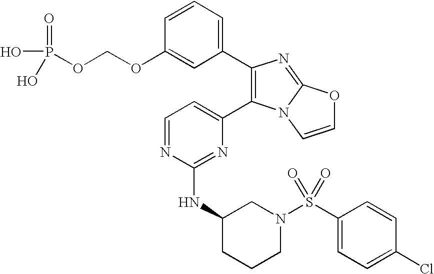 | (R)-(3-(5-(2-(1-(4- chlorophenylsulfonyl)piper- idin-3-ylamino)pyrimidin- 4-yl)imidazo[2,1-b]oxazol- 6-yl)phenoxy)methyl dihydrogen phosphate | 661 | 1.41 | 0.012 |
| 7 | 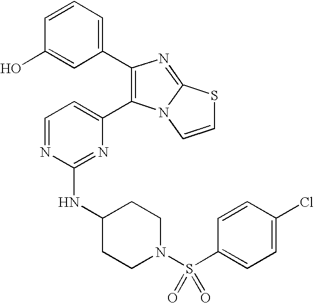 | 3-(5-(2-(1-(4- chlorophenylsulfonyl)piper- idin-4-ylamino)pyrimidin- 4-yl)imidazo[2,1-b]thiazol- 6-yl)phenol | 567 | 0.008 | 0.082 |
| 8 | 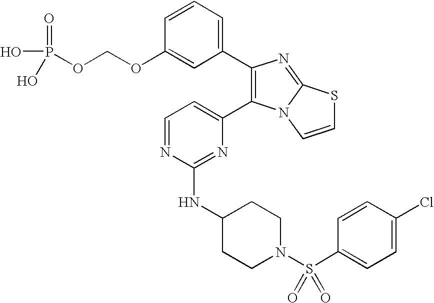 | (3-(5-(2-(1-(4- chlorophenylsulfonyl)piper- idin-4-ylamino)pyrimidin- 4-yl)imidazo[2,1-b]thiazol- 6-yl)phenoxy)methyl dihydrogen phosphate | 677 | 0.405 | 0.938 |
| 9 | 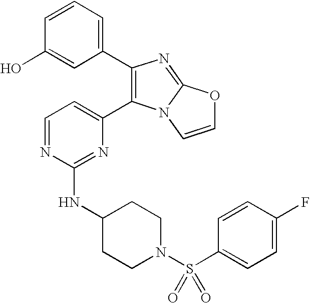 | 3-(5-(2-(1-(4- fluorophenylsulfonyl)piper- idin-4-ylamino)pyrimidin- 4-yl)imidazo[2,1-b]oxazol- 6-yl)phenol | 535 | 0.048 | 0.129 |
| 10 | 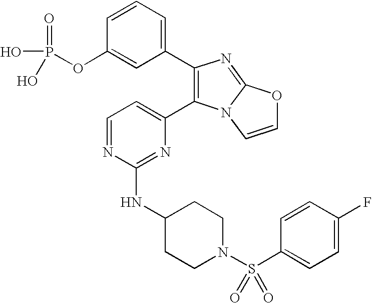 | 3-(5-(2-(1-(4- fluorophenylsulfonyl)piper- idin-4-ylamino)pyrimidin- 4-yl)imidazo[2,1-b]oxazol- 6-yl)phenyl dihydrogen phosphate | 615 | 1.17 | 0.088 |
| 11 | 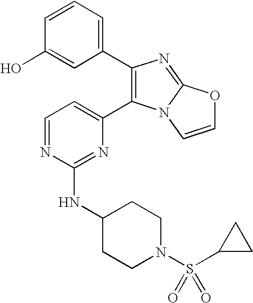 | 3-(5-(2-(1- (cyclopropylsulfonyl)piper- idin-4-ylamino)pyrimidin- 4-yl)imidazo[2,1-b]oxazol- 6-yl)phenol | 481 | 0.048 | 0.053 |
| 12 | 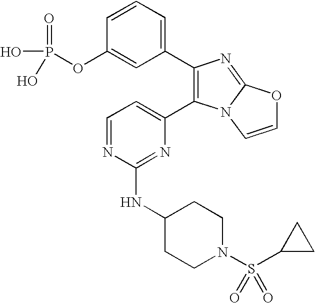 | 3-(5-(2-(1- (cyclopropylsulfonyl)piper- idin-4-ylamino)pyrimidin- 4-yl)imidazo[2,1-b]oxazol- 6-yl)phenyl dihydrogen phosphate | 561 | >3.3 | 0.177 |
| 13 | 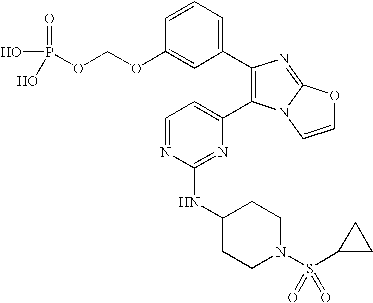 | (3-(5-(2-(1- (cyclopropylsulfonyl)piper- idin-4-ylamino)pyrimidin- 4-yl)imidazo[2,1-b]oxazol- 6-yl)phenoxy)methyl dihydrogen phosphate | 591 | >3.3 | 0.161 |
| 14 | 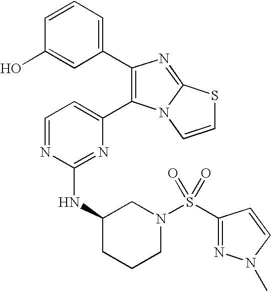 | (R)-3-(5-(2-(1-(1-methyl- 1H-pyrazol-3- ylsulfonyl)piperidin-3- ylamino)pyrimidin-4- yl)imidazo[2,1-b]thiazol- 6-yl)phenol | 537 | 0.006 | 0.002 |
| 15 | 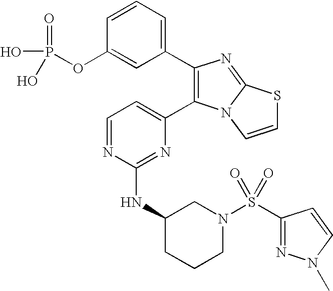 | (R)-3-(5-(2-(1-(1-methyl- 1H-pyrazol-3- ylsulfonyl)piperidin-3- ylamino)pyrimidin-4- yl)imidazo[2,1-b]thiazol- 6-yl)phenyl dihydrogen phosphate | 617 | 2.41 | 0.008 |
| 16 | 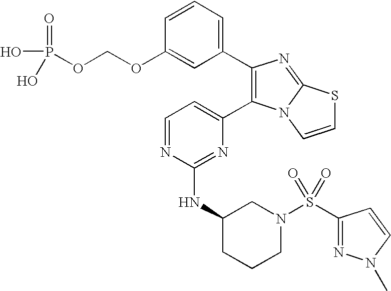 | (R)-(3-(5-(2-(1-(1-methyl- 1H-pyrazol-3- ylsulfonyl)piperidin-3- ylmino)pyrimidin-4- yl)imidazo[2,1-b]thiazol- 6-yl)phenoxy)methyl dihydrogen phosphate | 647 | 1.16 | 0.004 |
| 17 | 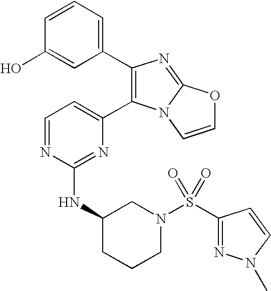 | (R)-3-(5-(2-(1-(1-methyl- 1H-pyrazol-3- ylsulfonyl)piperidin-3- ylamino)pyrimidin-4- yl)imidazo[2,1-b]oxazol- 6-yl)phenol | 521 | 0.008 | 0.093 |
| 18 | 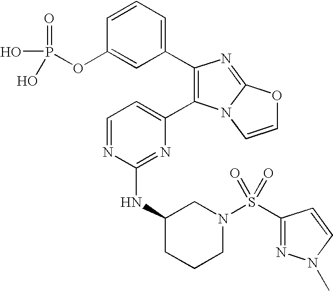 | (R)-3-(5-(2-(1-(1-methyl- 1H-pyrazol-3- ylsulfonyl)piperidin-3- ylamino)pyrimidin-4- yl)imidazo[2,1-b]oxazol- 6-yl)phenyl dihydrogen phosphate | 601 | >3.3 | 0.087 |
| 19 | 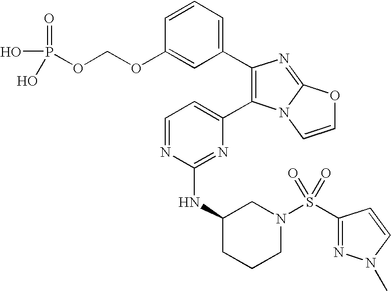 | (R)-(3-(5-(2-(1-(1- methyl-1H-pyrazol-3- ylsulfonyl)piperidin-3- ylamino)pyrimidin-4- yl)imidazo[2,1-b]oxazol- 6-yl)phenoxy)methyl dihydrogen phosphate | 631 | 2.92 | 0.095 |
| 20 | 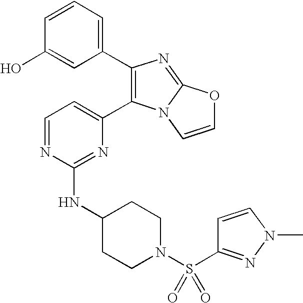 | 3-(5-(2-(1-(1-methyl- 1H-pyrazol-3- ylsulfonyl)piperidin-4- ylamino)pyrimidin-4- yl)imidazo[2,1-b]oxazol- 6-yl)phenol | 521 | 0.013 | 0.073 |
| 21 | 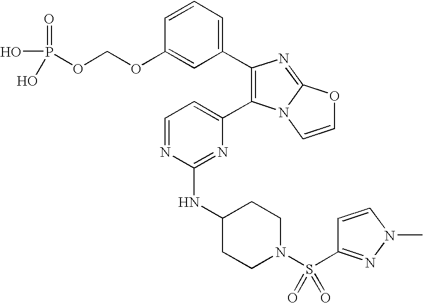 | (3-(5-(2-(1-(1-methyl- 1H-pyrazol-3- ylsulfonyl)piperidin-4- ylamino)pyrimidin-4- yl)imidazo[2,1-b]oxazol- 6-yl)phenoxy)methyl dihydrogen phosphate | 631 | 1.16 | 0.141 |
| 22 | 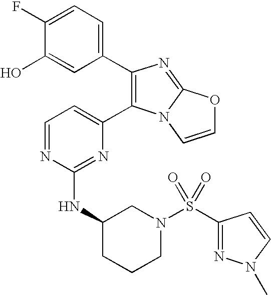 | (R)-2-fluoro-5-(5-(2-(1-(1- methyl-1H-pyrazol-3- ylsulfonyl)piperidin-3- ylamino)pyrimidin-4- yl)imidazo[2,1-b]oxazol- 6-yl)phenol | 539 | 0.00096 | 0.034 |
| 23 | 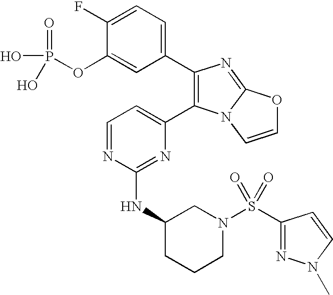 | (R)-2-fluoro-5-(5-(2-(1-(1- methyl-1H-pyrazol-3- ylsulfonyl)piperidin-3- ylamino)pyrimidin-4- yl)imidazo[2,1-b]oxazol- 6-yl)phenyl dihydrogen phosphate | 619 | 1.05 | 0.009 |
| 24 | 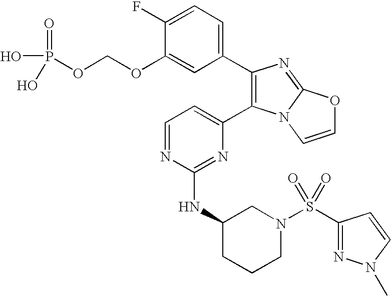 | (R)-(2-fluoro-5-(5-(2-(1- (1-methyl-1H-pyrazol-3- ylsulfonyl)piperidin-3- ylamino)pyrimidin-4- yl)imidazo[2,1-b]oxazol- 6-yl)phenoxy)methyl dihydrogen phosphate | 649 | 0.028 | 0.052 |

TABLE 2
Activity against cancer cell lines for some exemplary
Compounds of the Invention

| | GI₅₀ (μM) | | | | | |
|---|---|---|---|---|---|---|
| | SK- | | | | SK- | WM- |
| | MEL-28 | RKO | A375 | DLD-1 | MEL-2 | 266-4 |
| Compound 4 | 1.12 | 1.81 | 0.00054 | 15.4 | 1.8 | 0.015 |
| Compound 7 | 0.088 | 0.085 | <0.0004 | 5.52 | 0.519 | 0.006 |
| Compound 9 | 0.3 | 0.283 | <0.0004 | 1.62 | 0.356 | <0.0004 |
| Compound 11 | 0.555 | 0.323 | <0.0004 | 13.9 | 2.04 | 0.003 |
| Compound 14 | 0.074 | 0.122 | 0.002 | 3 | 0.17 | 0.005 |
| Compound 17 | 0.097 | 0.095 | <0.0004 | 12.1 | 0.154 | 0.0006 |
| Compound 20 | 0.393 | 0.372 | <0.0004 | 9.47 | 0.31 | 0.002 |
| Compound 22 | 0.033 | 0.052 | <0.0004 | 5.92 | 0.0428 | <0.0004 |

TABLE 3
Activity against cancer cell lines for Compound 19

| Cell line | Cell type | GI₅₀ (μM) |
|---|---|---|
| WM-266.4 | melanoma | 0.13 |
| THP-1 | acute monocytic leukemia | 10.31 |
| KG-1a | AML | 11.45 |
| KG-1 | AML | 30.80 |
| SW780 | bladder | 9.10 |
| MCF-7 | breast adenocarcinoma | 12.54 |
| MDAMB-231 | breast adenocarcinoma | 28.17 |
| K562 | CML | 9.86 |
| COLO-205 | colon | 0.25 |
| HCT-116 | colon | 6.61 |
| SW480 | colon | 8.27 |
| DLD-1 | colon | 19.00 |
| HCT-15 | colon | 23.23 |
| RKO | colon carcinoma | 0.57 |
| WIDR | colorectal adenocarcinoma | 20.22 |
| SW620 | colorectal adenocarcinoma | 22.77 |
| LS411N | colorectal carcinoma | 0.31 |
| LOVO | colorectal carcinoma | 2.17 |
| HT29 | colorectal carcinoma | 5.03 |
| LS174T | colorectal carcinoma | 5.90 |
| HEC1A | endometrial | 2.72 |
| AN3CA | endometrial | 20.05 |
| HT-1080 | fibrosarcoma | 26.83 |
| Kato111 | gastric carcinoma | 6.11 |
| SNU-16 | gastric Carcinoma | 15.70 |
| HEP-G2 | hepatocellular carcinoma | 1.38 |
| RT112 | human urinary bladder cell carcinoma | 1.92 |
| RT4 | human urinary bladder cell carcinoma | 10.01 |
| 786-O | kidney | 18.20 |
| CAKI-2 | kidney | 26.25 |
| CAKI-1 | kidney | 30.33 |
| NCI-H661 | large cell lung carcinoma | 16.24 |
| NCI-H460 | large cell lung carcinoma | 19.30 |
| SK-LMS-1 | leiomyosarcoma | 15.25 |
| HEP-3B | liver | 3.16 |
| PLC/PRF/5 | liver | 13.55 |
| SNU475 | liver | 29.05 |
| SK-HEP-1 | liver adenocarcinoma | 7.22 |
| CALU-6 | lung | 2.97 |
| NCI-H1993 | lung adenocarcinoma | 26.25 |
| A427 | lung Carcinoma | 3.23 |
| NCI-H526 | lung carcinoma, variant small cell lung | 58.86 |
| | cancer | |
| NCI-H441 | lung papillary adenocarcinoma | 17.84 |
| BDCM | lymphoblast | 32.71 |
| SK-MEL-28 | malignant melanoma | 1.70 |
| A-375 | melanoma | 0.28 |
| CC5292 | MiT | 8.20 |
| U937 | monocyte histocytic lymphoma | 20.17 |
| NCI-H358 | non-small cell lung cancer, | 7.50 |
| | brancioalveolar carcinoma | |
| NCI-H1299 | non-small cell lung carcinoma | 6.38 |
| SK-OV-3 | ovary | 13.69 |
| HPAF-II | pancreas | 5.78 |
| ASPC-1 | pancreas | 6.33 |
| PANC-1 | pancreas epithelioid carcinoma | 29.72 |
| CFPPAC-1 | pancreasductal adenocarcinoma; cystic | 11.88 |
| | fibrosis | |
| DU145 | prostate | 9.62 |
| A375 | skin | 0.30 |
| SKMES-1 | squamous cell carcinoma | 9.02 |
| NCI-H520 | squamous cell lung carcinoma | 20.25 |
| MKN-45 | stomach | 21.42 |
| NTERA-2 c1. D1 | testis pluripotent embryonal carcinoma | 5.32 |

TABLE 4
Aqueous solubility of exemplary Compounds at varying pH

| Aqueous solubility | compound 19 | compound 17 |
|---|---|---|
| pH 1.2 (mg/ml) | <1.3 | 1.05 |
| pH 3.0 (mg/ml) | not tested | 0.01 |
| pH 5.0 (mg/ml) | >13 | insoluble |
| pH 7.4 (mg/ml) | >300 | insoluble |

TABLE 5
Isobologram for Combinations of (R)-(3-(5-(2-(1-(1-methyl-1H-pyrazol-3-
ylsulfonyl)piperidin-3-ylamino)pyrimidin-4-yl)imidazo[2,1-b]oxazol-6-
yl)phenoxy)methyl dihydrogen phosphate with (−)-trans-3-(5,6-dihydro-
4H-pyrrolo [3,2,1-ij] quinolin-1-yl)-4(1H-indol-3-yl) pyrrolidine-2,5-dione

| | | Combination | |
|---|---|---|---|
| Cell Lines | Tissue Origin | Index | Classification |
| MDAMB-231 | Breast | 0.62 | Synergism |
| NCI-H520 | Lung (NSCLC) | 0.72 | Synergism |
| SNU475 | Liver | 0.73 | Synergism |
| WIDR | Colon | 0.76 | Synergism |
| NCI-H1993 | Lung (NSCLC) | 0.77 | Synergism |
| SNU-387 | Liver | 0.78 | Synergism |
| BX-PC3 | Pancreas | 0.84 | Synergism |
| WM-266.4 | Skin | 0.85 | Synergism |
| NCI-H661 | Lung (NSCLC) | 0.90 | Additive |
| SK-MES-1 | Lung (NSCLC) | 0.91 | Additive |
| A549 | Lung (NSCLC) | 0.92 | Additive |
| NCI-H460 | Lung (NSCLC) | 0.94 | Additive |
| NCI-H358 | Lung (NSCLC) | 0.95 | Additive |
| CAKI-2 | Kidney | 0.95 | Additive |
| NCI-H526 | Lung (NSCLC) | 0.95 | Additive |
| SNU-16 | Stomach | 0.95 | Additive |
| SW480 | Colon | 0.96 | Additive |
| U937 | Blood | 0.98 | Additive |
| HCT-15 | Colon | 0.99 | Additive |
| ASPC-1 | Pancreas | 0.99 | Additive |
| DU4475 | Breast | 1.00 | Additive |
| NCI-H1299 | Lung (NSCLC) | 1.02 | Additive |
| MKN-45 | Stomach | 1.03 | Additive |
| CCS292 | Tandon | 1.03 | Additive |
| PLC/PRF/5 | Liver | 1.03 | Additive |
| HCT-116 | Colon | 1.03 | Additive |
| 786-O | Kidney | 1.04 | Additive |
| HT29 | Colon | 1.06 | Additive |
| CALU-6 | Lung (NSCLC) | 1.07 | Additive |
| SNU398 | Liver | 1.07 | Additive |
| HEP-3B | Liver | 1.10 | Additive |
| PANC-1 | Pancreas | 1.10 | Additive |
| DU145 | Prostate | 1.10 | Additive |
| CAKI-1 | Kidney | 1.13 | Additive |
| THP-1 | Blood | 1.17 | Additive |
| HT-1080 | Connective Tissue | 1.17 | Additive |
| K562 | Blood | 1.21 | Antagonism |
| LS174T | Colon | 1.25 | Antagonism |
| Kato111 | Stomach | 1.28 | Antagonism |
| LS411N | Colon | 1.30 | Antagonism |
| SW780 | Bladder | 1.32 | Antagonism |
| A375 | Skin | 1.35 | Antagonism |
| RT112 | Bladder | 1.35 | Antagonism |
| SK-OV-3 | Ovary | 1.37 | Antagonism |
| SW620 | Colon | 1.48 | Antagonism |
| DLD-1 | Colon | 1.51 | Antagonism |
| RT4 | Bladder | 1.69 | Antagonism |
| COLO-205 | Colon | 1.71 | Antagonism |
| HL-60 | Blood | 1.73 | Antagonism |
| AN3CA | Uterus | 1.75 | Antagonism |
| HPAF-II | Pancreas | 1.91 | Antagonism |
| BDCM | Lung | 1.97 | Antagonism |
| SK-HEP-1 | Liver | 2.00 | Antagonism |
| SK-LMS-1 | Vulva | 2.13 | Antagonism |
| RKO | Colon | 2.75 | Antagonism |

Although the above discussion discloses various exemplary embodiments of the invention, it should be apparent that those skilled in the art can make various modifications that will achieve some of the advantages of the invention without departing from the true scope of the invention.

1. A compound of formula I, or pharmaceutically acceptable salts thereof: 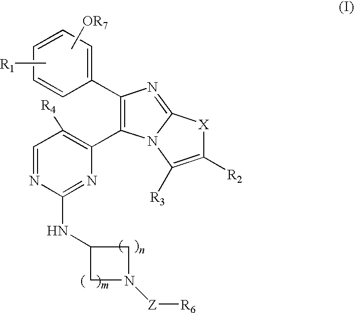
wherein
X is O, S(O)ₚ;
m is an integer from 1 to 3;
n is an integer from 1 to 3;
o is an integer from 0 to 2;
p is an integer from 0 to 2;
Z is hydrogen, a bond, —C(O)—, —C(O)NR₄—, —S(O)₂—;
R₁ is hydrogen, halogen, substituted or unsubstituted alkyl, —CN, —COOR₄, —OR₄, —NR₄R₅,
R₂ and R₃ are independently hydrogen, substituted or unsubstituted lower alkyl, —COOR₄, or —C(O)NR₄R₅;
each R₄ and each R₅ are independently hydrogen, substituted or unsubstituted alkyl, substituted or unsubstituted aryl, or substituted or unsubstituted heterocyclyl, and R₄ and R₅, taken together, may form a ring;
R₆ is independently selected from the group consisting of hydrogen, C₁-C₈ alkyl, C₁-C₈ fluoro-substituted alkyl, C₃-C₈ cycloalkyl, C₃-C₈ fluoro-substituted cycloalkyl, heterocyclyl, (C₁-C₈) alkyl-substituted heterocyclyl, aryl, halogen-substituted aryl, heteroaryl, (C₁-C₈) alkyl-substituted heteroaryl, and halogen-substituted heteroaryl;
R₇ is H or (CH₂O)ₒ—P(O)OR₄OR₅.

2. The compound of claim 1, wherein R₂ and R₃ are hydrogen.

3. The compound of claim 1, wherein R₄ is hydrogen.

4. The compound of claim 1, wherein m+n=4, if m is not equal to n, then the preferred configuration is R.

5. The compound of claim 1, wherein Z is hydrogen, a bond, —C(O)—, —C(O)NR₄—, —S(O)₂—; and R₆ is alkyl-substituted heterocyclyl, or alkyl-substituted heteroaryl.

6. A compound selected from the group consisting of (R)-3-(5-(2-(1-(4-chlorophenylsulfonyl)piperidin-3-ylamino)pyrimidin-4-yl)imidazo[2,1-b]oxazol-6-yl)phenyl dihydrogen phosphate; (R)-3-(5-(2-(1-(4-chlorophenylsulfonyl)piperidin-3-ylamino)pyrimidin-4-yl)imidazo[2,1-b]thiazol-6-yl)phenyl dihydrogen phosphate; (R)-(3-(5-(2-(1-(4-chlorophenylsulfonyl)piperidin-3-ylamino)pyrimidin-4-yl)imidazo[2,1-b]thiazol-6-yl)phenoxy)methyl dihydrogen phosphate; (R)-(3-(5-(2-(1-(4-chlorophenylsulfonyl)piperidin-3-ylamino)pyrimidin-4-yl)imidazo[2,1-b]oxazol-6-yl)phenoxy)methyl dihydrogen phosphate; (R)-((3-(5-(2-(1-(4-chlorophenylsulfonyl)piperidin-3-ylamino)pyrimidin-4-yl)imidazo[2,1-b]thiazol-6-yl)phenoxy)methoxy)methyl dihydrogen phosphate; (3-(5-(2-(1-(4-chlorophenylsulfonyl)piperidin-4-ylamino)pyrimidin-4-yl)imidazo[2,1-b]thiazol-6-yl)phenoxy)methyl dihydrogen phosphate; (3-(5-(2-(1-(4-cyanophenylsulfonyl)piperidin-4-ylamino)pyrimidin-4-yl)imidazo[2,1-b]oxazol-6-yl)phenoxy)methyl dihydrogen phosphate; 3-(5-(2-(1-(4-fluorophenylsulfonyl)piperidin-4-ylamino)pyrimidin-4-yl)imidazo[2,1-b]oxazol-6-yl)phenyl dihydrogen phosphate; (3-(5-(2-(1-(cyclopropylsulfonyl)piperidin-4-ylamino)pyrimidin-4-yl)imidazo[2,1-b]oxazol-6-yl)phenoxy)methyl dihydrogen phosphate; ((3-(5-(2-(1-(cyclopropylsulfonyl)piperidin-4-ylamino)pyrimidin-4-yl)imidazo[2,1-b]oxazol-6-yl)phenoxy)methoxy)methyl dihydrogen phosphate; (3-(5-(2-(1-(1-methyl-1H-pyrazol-3-ylsulfonyl)piperidin-4-ylamino)pyrimidin-4-yl)imidazo[2,1-b]oxazol-6-yl)phenoxy)methyl dihydrogen phosphate; (R)-3-(5-(2-(1-(1-methyl-1H-pyrazol-3-ylsulfonyl)piperidin-3-ylamino)pyrimidin-4-yl)imidazo[2,1-b]oxazol-6-yl)phenyl dihydrogen phosphate; (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate; (R)-((3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methoxy)methyl dihydrogen phosphate; (R)-(3-(5-(2-(1-(1-methyl-1H-pyrazol-3-ylsulfonyl)piperidin-3-ylamino)pyrimidin-4-yl)imidazo[2,1-b]thiazol-6-yl)phenoxy)methyl dihydrogen phosphate; (R)-2-fluoro-5-(5-(2-(1-(1-methyl-1H-pyrazol-3-ylsulfonyl)piperidin-3-ylamino)pyrimidin-4-yl)imidazo[2,1-b]oxazol-6-yl)phenyl dihydrogen phosphate; and (R)-(2-fluoro-5-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate or a pharmaceutically acceptable salt thereof.

7. A compound selected from the group consisting of (R)-3-(5-(2-(1-(4-chlorophenylsulfonyl)piperidin-3-ylamino)pyrimidin-4-yl)imidazo[2,1-b]thiazol-6-yl)phenyl dihydrogen phosphate; (R)-(3-(5-(2-(1-(4-chlorophenylsulfonyl)piperidin-3-ylamino)pyrimidin-4-yl)imidazo[2,1-b]oxazol-6-yl)phenoxy)methyl dihydrogen phosphate; and (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate or a pharmaceutically acceptable salt thereof.

8. The compound (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate or a pharmaceutically acceptable salt thereof.

9. A prodrug, wherein the prodrug is hydrolyzed in vivo to give a compound of formula I as defined by claim 1, wherein R₇ is H or CH₂OH after the hydrolysis.

10. A pharmaceutical composition comprising a compound as defined in claim 1 or a pharmaceutically acceptable salt thereof together with one or more pharmaceutically acceptable carriers or excipients.

11. The pharmaceutical composition of claim 10, further comprising a second chemotherapeutic agent.

12. The pharmaceutical composition of claim 10, wherein said second chemotherapeutic agent is selected from the group consisting of tamoxifen, raloxifene, anastrozole, exemestane, letrozole, cisplatin, carboplatin, paclitaxel, cyclophosphamide, lovastatin, mimosine, gemcitabine, Ara, 5-fluorouracil, methotrexate, docetaxel, goserelin, vincristine, vinblastine, nocodazole, teniposide, etoposide, epothilone, navelbine, camptothecin, daunorubicin, dactinomycin, mitoxantrone, amsacrine, doxorubicin, epirubicin, idarubicin imatanib, gefitinib, erlotinib, sorafenib, sunitinib malate, trastuzumab, rituximab, cetuximab, and bevacizumab.

13. The pharmaceutical composition of claim 10, wherein said second chemotherapeutic agent is selected from the group consisting of a taxane, an aromatase inhibitor, an anthracycline, a microtubule targeting drug, a topoisomerase poison drug, a targeted monoclonal or polyconal antibody, an inhibitor of a molecular target or enzyme (e.g., a kinase inhibitor), or a cytidine analogue drug.

14. The pharmaceutical composition of claim 10, wherein said second chemotherapeutic agent is (−)-trans-3-(5,6-dihydro-4H-pyrrolo[3,2,1-ij] quinolin-1-yl)-4(1H-indol-3-yl) pyrrolidine-2,5-dione.

15. The pharmaceutical composition of claim 14, wherein the compound of formula I is (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate.

16. A method of treating or preventing a cell proliferative disorder, said method comprising administering to a subject having cells with the cell proliferative disorder a therapeutically effective amount of a compound of formula I as defined by claim 1, or a pharmaceutically acceptable salt thereof in combination with a pharmaceutically acceptable carrier, wherein said cell proliferative disorder is treated.

17. The method of claim 16, wherein the cells with proliferative disorder contain DNA encoding a RAF.

18. The method of claim 16, wherein the RAF is A-RAF, B-RAF, or C-RAF.

19. The method of claim 17, wherein the RAF is B-RAF.

20. The method of claim 18, wherein the B-RAF is wild-type.

21. The method of claim 18, wherein the B-RAF is a mutant.

22. The method of claim 21, wherein the B-RAF mutant is B-RAF^{V600E}.

23. The method of claim 16, wherein the cells have a constitutively enhanced RAF activity.

24. The method of claim 16, wherein said cell proliferative disorder is a precancerous condition.

25. The method of claim 16, wherein said cell proliferative disorder is a cancer.

26. The method of claim 16, wherein said cell proliferative disorder is melanoma.

27. The method of claim 16, wherein said cell proliferative disorder is papillary thyroid cancers.

28. The method of claim 16, wherein said cell proliferative disorder is colon cancer.

29. The method of claim 16, wherein said cell proliferative disorder is one of breast cancer, lung cancer, colorectal cancer, pancreatic cancer, ovarian cancer, prostate cancer, renal carcinoma, hepatoma, brain cancer, melanoma, multiple myeloma, chronic myelogenous leukemia, hematologic tumor, lymphoid tumor, sarcoma, carcinoma, and adenocarcinoma.

30. The method of claim 16, wherein said cell proliferative disorder is Congenital Nevi.

31. The method of claim 16, wherein said compound or a pharmaceutically acceptable salt thereof is administered in combination with a second chemotherapeutic agent.

32. The method of claim 31, wherein said second chemotherapeutic agent is selected from the group consisting of tamoxifen, raloxifene, anastrozole, exemestane, letrozole, cisplatin, carboplatin, paclitaxel, cyclophosphamide, lovastatin, minocin, gemcitabine, Ara, 5-fluorouracil, methotrexate, docetaxel, goserelin, vincristin, vinblastin, nocodazole, teniposide, etoposide, epothilone, navelbine, camptothecin, daunorubicin, dactinomycin, mitoxantrone, amsacrine, doxorubicin, epirubicin, idarubicin imatanib, gefitinib, erlotinib, sorafenib, sunitinib malate, trastuzumab, rituximab, cetuximab, and bevacizumab.

33. The method of claim 31, wherein said second chemotherapeutic agent is (−)-trans-3-(5,6-dihydro-4H-pyrrolo[3,2,1-ij] quinolin-1-yl)-4(1H-indol-3-yl) pyrrolidine-2,5-dione.

34. The method of claim 32, wherein the compound of formula I is (R)-(3-(5-(2-(1-(1-methyl-1H-pyrazol-3-ylsulfonyl)piperidin-3-ylamino)pyrimidin-4-yl)imidazo[2,1-b]oxazol-6-yl)phenoxy)methyl dihydrogen phosphate.

35. The method of claim 33, wherein the cancer is a breast cancer, lung cancer, liver cancer, colon cancer or pancreatic cancer.
